(19) United States
(12) Patent Application Publication
(10) Pub. No.: US 2004/0033200 A1
(43) Pub. Date: Feb. 19, 2004

(54) Modified FVII in treatment of ARDS

(76) Inventors: Mirella Ezban, Kobenhavn K (DK); Claude A. Piantadosi, Durham, NC (US); Steven Idell, Tyler, TX (US)

Correspondence Address: Reza Green, Esq., Novo Nordisk Pharmaceuticals, Inc., 100 College Road West, Princeton, NJ, 08540, US

(21) Appl. No.: 10/283,482

(22) Filed: Oct. 30, 2002

Related U.S. Application Data
(63) Continuation of application No. PCT/DK02/00279, filed on May 1, 2002.

(30) Foreign Application Priority Data
May 2, 2001 (DK) PA 2001 00692

(51) Int. Cl. A61K 38/39; A61L 9/04

(52) U.S. Cl. 424/45; 514/12

(57) ABSTRACT

The present invention relates to the use of modified factor VI[ for manufacture of medicaments for treatment of Acute Lung Injury (ALI) or Acute Respiratory Distress Syndrome (ARDS) in humans.

### CROSS REFERENCE TO RELATED APPLICATIONS

This application is a continuation of PCT/DK02/00279 filed on May 1, 2002, and claims proiroty under 35 U.SC. 119 of Danish application no. PA 2001 00692 filed on May 2, 2001, the contents of which are fully incorporated herein by reference.

### FIELD OF INVENTION

The present invention relates to the use of modified FVII for the manufacture of medicaments for treatment of Acute Lung Injury (ALI) and Acute Respiratory Distress Syndrome (ARDS), and to a method for treating ALI and ARDS. The invention also relates to use of modified FVII for the manufacture of medicaments for preventing or minimizing chronic organ failure associated with ALI or ARDS, and for preventing or minimizing such chronic organ failure.

### BACKGROUND

Acute respiratory distress syndrome (“ARDS”) is a manifestation of the systemic inflammatory response syndrome (SIRS) that can develop e.g. in trauma patients. The syndrome is an acute illness, characterized by systemic inflammatory mediator release and generalized activation of the endothelium, eventually leading to multiple organ dysfunctions syndrome. Infectious insults (e.g. sepsis), as well as non-infectious pathologic causes (e.g. trauma and tissue injuries), can produce SIRS and manifest ARDS. ARDS is described as a “syndrome of inflammation and increased permeability associated with a constellation of clinical, radiological and physiological abnormalities” (Am-European Consensus from 1994). It develops as a complication to acute diseases or injuries such as sepsis, pneumonia, aspiration, ischemia (circulatory arrest, hemorrhagic shock), trauma and others. In patients with ARDS the microvascular, interstitial and alveolar spaces of the lungs are the primary targets for fibrin deposition. This is primarily due to the large surface area of the lung (70 m2) and the position of the pulmonary capillaries to receive the entire cardiac output. However, devastating micro thrombus formation occurs in multiple organs, with lungs and kidneys as the most exposed, which may lead to the development of multiple organ failure (MOF). Furthermore, the inflammatory response also results in vascular leakage of plasma proteins into the alveolar spaces of the lungs causing lung oedema.

The hallmark of ARDS is deterioration in blood oxygenation and respiratory system compliance as a consequence of permeability oedema. Whereas a variety of different insults may lead to ARDS, a common pathway probably results in the lung damage and/or failure, leukocyte activation within the lung, along with the release of oxygen free radicals, rachidonic acid metabolites, and inflammatory mediators such as interlueukin-1, proteases, and tumour necrosis factor results in an increase in alveolo-capillary membrane permeability. With the loss of this macromolecular barrier, alveoli is flooded with serum proteins, which impair the function of pulmonary surfactant (Said et al. J.Clin.lnvest. 44:458-464; Holm et al. J.Appl.Physiol. 63:1434-1442, 1987) This creates hydrostatic forces that further exacerbates the condition (Jefferies et al., J.Appl. Physiol. 64:5620-5628, 1988), leading to alveolar edema and a concomitant deterioration in gas exchange and lung compliance.

ARDS affects both medical and surgical patients. The syndrome is often progressive, characterized by distinct stages with different clinical, histopathological, and radiographic manifestations. The acute, or exudative, phase is manifested by the rapid onset of respiratory failure in a patient with a risk factor for the condition. Arterial hypoxemia that is refractory to treatment with supplemental oxygen is a characteristic feature. Radiographically, the findings are indistinguishable from those of cardiogenic pulmonary oedema. Bilateral infiltrates may be patchy or asymmetric and may include pleural effusions. Alveolar filling, consolidation, and atelectasis occur predominantly in dependent lung zones, whereas other areas may be relatively spared. However, even spared, nondependent areas may have substantial inflammation. Pathological findings include diffuse alveolar damage, with neutrophils, macrophages, erythrocytes hyaline membranes, and protein-rich oedema fluid in the alveolar spaces, capillary injury, and disruption of the alveolar epithelium.

Although acute lung injury and ARDS may resolve completely in some patients after the acute phase, in others it progresses to fibrosing alveolitis with persistent hypoxemia, increased alveolar dead space, and a further decrease in alveolar or pulmonary compliance. Pulmonary hypertension, owing to obliteration of the pulmonary-capillary bed, may be severe and lead to right ventricular failure. In most patients who survive ARDS, pulmonary function returns to nearly normal within 6-12 months, despite the severe injury to the lung. Residual impairment of pulmonary mechanics may include mild restriction, obstruction, impairment of the diffusing capacity for carbon monoxide, or gas-exchange abnormalities with exercise, but these abnormalities are usually asymptomatic. Severe disease and prolonged mechanical ventilation identify patients at highest risk for persistent abnormalities of pulmonary function. Those who survive the illness have a reduced health-related quality of life as well as pulmonary-disease-specific health-related quality of life.

Most studies of ALI and ARDS have reported a mortality rate of 40-60%. The majority of deaths are attributable to sepsis or multiorgan dysfunction rather than primary respiratory causes, although the recent therapeutic success of ventilation with low tidal volumes indicates that in some cases death is directly related to lung injury.

In 1988, an expanded definition of the syndrome was proposed that quantified the physiological respiratory impairment through the use of a four-point lung-injury scoring system that was based on the level of positive and expiratory pressure, the ratio of the partial pressure of arterial oxygen to the fraction of inspired oxygen, the static lung compliance, and the degree of infiltration evident on chest radiographs. Other factors included in the assessments were the inciting clinical disorder and the presence or absence of nonpulmonary organ dysfunction. In 1994, a new definition was recommended by the American-European Consensus Conference Committee: First, it recognizes that the severity of clinical lung injury varies: patients with less severe hypoxemia (as defined by a ratio of the partial pressure of arterial oxygen to the fraction of inspired oxygen of 300 or less) are considered to have ALI, and those with more severe hypoxemia (as defined by a ratio of 200 or less) are considered to have the ARDS. Second, the definition is simple to apply in the clinical setting. The widespread acceptance of both the 1994 consensus definition and the 1988 lung-injury scoring system has improved the standardization of clinical research and trials.

In consequence, acute lung injury (ALI) is defined by the following criteria ((Bernard et al., Am.J.Respir.CritCare Med 149: 818-24, 1994):

Acute onset

Bilateral infiltrates on chest radiography

Pulmonary-artery wedge pressure is ≦18 mm Hg or the absence of clinical evidence of left atrial hypertension

PaO₂:FiO₂ is ≦300

ARDS is defined by the following criteria (Bernard et al., Am.J.Respir.Crit Care Med 149: 818-24, 1994):

Acute onset

Bilateral infiltrates on chest radiography

Pulmonary-artery wedge pressure is ≦18 mm Hg or the absence of clinical evidence of left atrial hypertension

PaO₂:FiO₂ is ≦200

(PaO₂ denotes partial pressure of arterial oxygen, and FiO₂ fraction of inspired oxygen)

ARDS may be triggered by clinical disorders associated with direct injury to the lung and those that cause indirect lung injury in the setting of a systemic process (see Table A):

TABLE A
Clinical disorders associated with the development of ARDS

| Direct lung injury | Indirect lung injury |
|---|---|
| Common causes: | Common causes: |
| Pneumonia | Sepsis |
| Aspiration of gastric contents | Severe trauma with shock and |
| | multiple transfusions |
| Less common causes: | Less common causes: |
| Pulmonary contusion | Cardiopulmonary by-pass |
| Fat emboli | Drug overdoes |
| Near-drowning | Acute pancreatitis |
| Inhalational injury | Transfusion of blood products |
| Reperfusion pulmonary oedema | |

Overall, sepsis is associated with the highest risk of progression to ARDS, about 40%.

Diseases such as sepsis, that change how inflammation is regulated, cause severe ALI due to inappropriate and/or excessive stimulation of host defences. During inflammation, several components of the extrinsic coagulation pathway, including tissue factor (TF), activated factors VII (FVIIa) and X (FXa) and thrombin, interact with key inflammatory mediators to regulate tissue responses. Activation of coagulation occurs rapidly after infusion of endotoxin or bacteria with the development of a pro-coagulant environment in the vascular space. These changes are TF dependent and associated with increases in inflammatory cytokines. Likewise in the lung, a pro-coagulant state has been measured in animals after endotoxin infusion or with experimental ALI. A similar pro-coagulant environment has been found in the bronchoalveolar lavage (BAL) of patients with ARDS, suggesting that extravascular lung inflammation also activates the extrinsic pathway. Although inflammatory mediators have specific effect upon coagulation, the converse relationship of the role of TF, and related events in coagulation as regulatory factors in inflammatory responses, is less well understood.

There is a need in the art for medicaments useful in the treatment of ALI or ARDS. We have found that Modified FVII attenuates both the inflammatory and the coagulopathic responses in the course of the development of acute lung injury, and that blockade of coagulation with Modified FVII in subjects with established ALI or ARDS attenuates lung and renal injury and preserves lung and kidney function. Other tissues were also protected. Blocking of TF/FVIIa activity by Modified FVII in a model of established acute lung injury significantly and dramatically prolonged survival and attenuated the inflammatory and coagulopathic responses. This was evidenced by data showing an essential prevention of fibrin deposition in lungs, kidneys and other organs, preservation of organ function and a significant attenuation of IL-6 and IL-8 release.

### SUMMARY OF INVENTION

In one aspect, the invention provides the use of modified FVII for the manufacture of a medicament for treatment of Acute Lung Injury (ALI) or Acute Respiratory Disease Syndrome (ARDS) in humans.

In one embodiment, the invention provides the use of modified FVII for the manufacture of a medicament for treatment of symptoms and conditions associated with Acute Lung Injury (ALI) or Acute Respiratory Disease Syndrome (ARDS) in humans.

In one embodiment the medicament is for treatment of organ failure.

In one embodiment the medicament is for preventing failure of additional organs.

In one embodiment the medicament is for maintaining or improving organ function. In one embodiment the medicament is for treatment of pulmonary hypertension. In one embodiment the medicament isfor decreasing or minimizing procoagulant activity. In one embodiment thereof the procoagulant activity is associated with tissue factor expression by lung epithelial cells and tissue macrophages. In one embodiment the medicament is for decreasing or minimizing inflammation. In one embodiment the medicament is for decreasing or minimizing production of IL-6 and IL-8. In one embodiment the medicament is for improving pulmonary gas exchange. In one embodiment the medicament is for decreasing or minimizing lung oedema. In one embodiment the medicament is for decreasing or minimizing lung protein leakage.

In another aspect, the invention provides the use of modified FVII for the manufacture of a medicament for preventing or minimizing chronic organ failure associated with ALI or ARDS in humans. In one embodiment the ALI or ARDS is established before modified FVII is administered.

In one embodiment of the invention the organ is kidney, lung, adrenals, liver, small bowel, cardiovascular system, or haemostatic system. In one embodiment the organ is lung. In one embodiment the organ is kidney. In one embodiment the organ is the cardiovascular system. In one embodiment the organ is the haemostatic system.

In one aspect, the invention provides a method for treating Acute Lung Injury (ALI) or Acute Respiratory Disease Syndrome (ARDS) in humans, the method comprising administring a therapeutically effective amount of modified FVII to the subject in need of such treatment.

In different embodiments of the invention the method is for treating organ failure, for preventing failure of additional organs, treatment of pulmonary hypertension, decreasing or minimizing procoagulant activity, decreasing or minimizing inflammation, decreasing or minimizing production of IL-6 and IL-8, improving pulmonary gas exchange, decreasing or minimizing lung oedema, and decreasing or minimizing lung protein leakage.

In one aspect, the invention provides a method for preventing or minimizing chronic organ failure associated with ALI or ARDS in humans, the method comprising administring a therapeutically effective amount of modified FVII to the subject in need of such treatment. In one embodiment the ALI or ARDS is established before modified FVII is administered.

In an additional aspect, the invention provides the use of FVIIai for the manufacture of a medicament for treatment of lung failure. In one embodiment the lung damage is acute lung injury (ALI). In one embodiment the lung damage is acute respiratory distress syndrome (ARDS). In one embodiment the treatment of lung damage is preventing ALI from developing into ARDS. In a further aspect the invention provides the use of FVIIai for the manufacture of a medicament for protecting against further lung damage in established ALI or ARDS. In a further aspect the invention provides the use of FVIIai for the manufacture of a medicament for maintaining or improving lung function in established ALI and ARDS. In one aspect the invention provides a method for treating lung damage in a subject, the method comprising administring a therapeutically effective amount of FVIIai to the subject in need of such treatment. In one embodiment the lung damage is acute lung injury (ALI). In one embodiment the lung damage is acute respiratory distress syndrome (ARDS). In one embodiment the treatment of lung damage is preventing ALI from developing into ARDS. In a further aspect the invention provides a method for protecting against further lung damage in a subject having established ALI or ARDS, the method comprising administering a therapeutically effective amount of FVIIai to the subject in need of such treatment. In a further aspect the invention provides a method for maintaining or improving lung function in a subject having established ALI or ARDS, the method comprising administering a therapeutically effective amount of FVIIai to the subject in need of such treatment. In one further aspect, the invention provides the use of modified FVII for the manufacture of a medicament for treatment of pulmonary hypertension. In another aspect, the invention provides a method for treatment of pulmonary hypertension in a subject, the method comprising administering a therapeutically effective amount of modified FVII to the subject in need of such a treatment. In one embodiment, the pulmonary hypertension is associated with acute lung injury (ALI); in another embodiment, the pulmonary hypertension is associated with acute respiratory disease syndrome (ARDS). In another aspect, the invention provides the use of modified FVII for the manufacture of a medicament for decreasing or inhibiting procoagulant activity in the lung. In another aspect, the invention provides a method for decreasing or inhibiting procoagulant activity in the lung of a subject, the method comprising administering a therapeutically effective amount of modified FVII to the subject in need of such a treatment. In one embodiment, the procoagulant activity is associated with tissue factor expression by lung epithelial cells and tissue macrophages. In one aspect, the invention provides the use of modified FVII for the manufacture of a medicament for decreasing or inhibiting extravascular fibrin deposition. In another aspect, the invention provides a method for decreasing or inhibiting extravascular fibrin deposition in a subject, the method comprising administering a therapeutically effective amount of modified FVII to the subject in need of such a treatment. In one embodiment, the extravascular fibrin deposition is deposition in the lung. In one embodiment, the extravascular fibrin deposition is deposition during organ injury. In one aspect, the invention provides the use of modified FVII for the manufacture of a medicament for decreasing or inhibiting lung inflammation. In another aspect, the invention provides a method for decreasing or inhibiting lung inflammation in a subject, the method comprising administering a therapeutically effective amount of modified FVII to the subject in need of such a treatment.

In one embodiment of the invention the modified FVII is FVII having at least one amino acid residue substitution, insertion, or deletion in the catalytic triad. In one embodiment the modified FVII is FVII having at least one amino acid residue substitution, insertion, or deletion in positions Ser₃₄₄, Asp₂₄₂, and His₁₉₃ (positions referring to sequence of wild-type human FVII as described in U.S. Pat. No. 4,784,950). In one embodiment the active site residue Ser₃₄₄ is modified, replaced with Gly, Met, Thr, or more preferably, Ala. In one embodiment the modified FVII is FVIIa modified by reaction with a serine protease inhibitor. In one embodiment the protease inhibitor is an organophosphor compound, a sulfanyl fluoride, a peptide halomethyl ketone, or an azapeptide. In one embodiment the protease inhibitor is a peptide halomethyl ketone selected from Dansyl-L-Phe-Pro-Arg chloromethyl ketone, Dansyl-L-Glu-Gly-Arg chloromethyl ketone, Dansyl-L-Phe-Phe-Arg chloromethyl ketone and L-Phe-Phe-Arg chloromethylketone, Dansyl-D-Phe-Pro-Arg chloromethyl ketone, Dansyl-D-Glu-Gly-Arg chloromethyl ketone, Dansyl-D-Phe-Phe-Arg chloromethyl ketone and D-Phe-Phe-Arg chloromethylketone. In one embodiment the protease inhibitor is D-Phe-Phe-Arg chloromethylketone.

In one embodiment the modified Factor VII has less than about 5% of the catalytic activity of wild-type Factor VII of the corresponding species, more preferably less than about 1%.

In one embodiment ALI or ARDS has been induced by sepsis; in one ambodiment the ALI or ARDS has been induced by trauma.

In one embodiment the invention provides the use of modified FVII for the manufacture of a medicament for treatment of established Acute Lung Injury (ALI) or established Acute Respiratory Disease Syndrome (ARDS) in humans.

In one embodiment, the Modified FVII is administered as one or more bolus injections.

In one embodiment Modified FVII is administered in an amount of from about 0.05 mg to 500 mg/day; 1 mg to 200 mg/day; 1 mg to about 150 mg/day; 1 mg to about 125 mg/day; 1 mg to about 100 mg/day; 10 mg to about 175 mg/day; 10 mg to about 150 mg/day; or 10 mg to about 125 mg/day for a 70 kg patient.

In one embodiment modified FVII is administered by way of multiple iv. Injections.

In one embodiment modified FVII is administered in doses per day (24 hours) of 100 μg/kg×1, 100 μg/kg×2, 100 μg/kg×4, 200 μg/kg×1, 200 μg/kg×2, 200 μg/kg×4, 400 μg/kg×1, 400 μg/kg×2, 400 μg/kg×4, 800 μg/kg×1, or 800 μg/kg×2. In one embodiment hereof, the modified FVII is administered to the patient for one day; in another embodiment the modified FVII is administered to the patient for two days; in another embodiment the modified FVII is administered to the patient for three days.

### BRIEF DESCRIPTION OF THE FIGURES

FIG. 1. Tissue factor (TF) expression in E.coli sepsis. Western blot (A) showed increased TF expression in the lungs of sepsis control animals compared to normal baboon lung that was prevented by treatment with FVIIai. One of the two animals treated with TFPI had no change in TF expression. A representative blot is shown. (B) Densitometry performed on the sepsis control and FVIIai treated groups and normalized to the mean of non-septic normal control animals. N=6 in the two experimental groups and n=3 for normal controls. Data shown is mean±sem(* p<0.05 vs. normal controls, δ p<0.05 vs. sepsis controls)

FIG. 2. Sepsis-induced lung injury was prevented by FVIIai. Data are shown as change from t=12 hours to show drug effect. Graphs also show data from two animals treated with TFPI and cumulative data from sepsis controls from this lab. -•- sepsis control group (n=6), -*- sepsis+FVIIai (n=6), . . . - cumulative sepsis control (n=11), - - - sepsis+TFPI (n=2). The data are shown as ±sem and were analyzed using two factor ANOVA (* p<0.05). FVIIai prevented (A) increased arterial-alveolar oxygen gradient (AaDO₂, p<0.0001), (B) decline in lung system compliance (Cs, p<0.001), and (C) increase in mean pulmonary artery pressure (PAM, p<0.001), and (D) pulmonary vascular resistance (PVR, p<0.05).

FIG. 3. FVIIai treatment decreased lung inflammation. Lung MPO activity and BAL LDH were decreased in treated animals compared to sepsis controls (Φ p=0.07 and * p<0.01). There was no difference in BAL protein between the two groups. Data are shown as mean±sem and were analyzed using t-test.

FIG. 4. Renal and metabolic indices of sepsis-induced injury were improved in FVIIai treated animals. (A) Serum [HCO₃] was higher in FVIIai treated septic animals (p<0.01). (B) Serum creatinine increased in the sepsis control group but not in the sepsis group treated with FVIIai (p=0.059). (C) and (D) show similar fluid balance (iv. fluids minus urine output) in the two groups but higher urine output during sepsis in animals treated with FVIIai (p<0.0001). The data are shown as mean ± sem and were analyzed using two factor ANOVA. -•- sepsis control group (n=6), -*- sepsis+FVIIai (n=6).

FIG. 5. FVIIai attenuated sepsis-induced coagulophathy. (A) Sepsis caused progressive prolongation of PTT that was decreased in animals treated with FVIIai, p<0.01. Fibrinogen depletion (B) and elevation in TAT complexes (C) were attenuated in the treatment group, p<0.0001 for both. ATIII activity (D) shown as % of the kit standard declined in both groups but the differences did not reach statistical significance. The data are shown as mean±sem and were analyzed using two factor ANOVA. -•- sepsis control group (n=6), -*- sepsis+FVIIai (n=6).

FIG. 6. Inflammatory cytokines in sepsis were attenuated by FVIIai. The data are shown as mean±sem and were analyzed using two factor ANOVA. -•- sepsis control group (n=6), -*- sepsis+FVIIai (n=6). Sepsis-induced increases in IL-6 (A), IL-8 (B), and TNFR-1 (D) levels were all attenuated by treatment with FVIIai, p<0.01 for all. IL-1β level (C) was unchanged by TF blockade.

### DETAILED DESCRIPTION

Abbreviations

| | |
|---|---|
| AaDO2 | arterial-alveolar oxygen gradient |
| APTT | Activated partial thromboplastin time |
| ALI | Acute lung injury |
| APC | Activated protein C |
| ARDS | Acute respiratory distress syndrome |
| ASIS | FFR-rFVIIa |
| BAL | Bronchoalveolar lavage |
| BUN | blood urea nitrogen |
| BW | Body weight |
| CO | cardiac output, L/min |
| Cs | decline in lung system compliance |
| DO2 | oxygen delievery, mL/min |
| DVT | Deep vein thrombosis |
| F1-2 | Fibrinogen fragment 1 & 2. |
| FiO2 | fraction inspired oxygen |
| FFR | D-phenylalanyl-L-phenylalanyl-L-arginyl-tripeptide |
| FFR-rFVIIa | FFR-inactivated, rFVIIa |
| FPA | Fibrinopeptide A |
| FVII | Human coagulation factor VII |
| FVIIa | Human activated coagulation factor VII |
| IL-1β | Interleukin-1 beta |
| IL-6 | Interleukin-6 |
| IL-8 | Interleukin-8 |
| Kg | Kilogram |
| LPS | Lipopolysaccharid |
| MW | Molecular weight |
| NIH | National Institute of Health |
| NOEL | No observed effect level |
| PAM | increase in mean pulmonary artery pressure |
| PaO2 | Oxygen tension of arterial blood |
| PCWP | pulmonary capillary wedge pressure, mm Hg |
| PT | Prothrombin time |
| PTCA | Percutaneous transluminar coronary angioplasty |
| PVR | pulmonary vascular resistance |
| RBC | Red blood cells |
| rFVIIa | Recombinant, activated human factor VII |
| SVR | systemic vascular resistance, dynes × cm × kg/10 |
| TAT | Thrombin-antithrombin complexes |
| TF | Tissue factor |
| TNFR-1 | TNF receptor-1 |
| TFPI | Tissue factor pathway inhibitor |
| VO2 | oxygen comsumption, mL/min |
| μg | Microgram |

The term “organ damage” encompasses, without limitation, damage to the structure and/or damage to the functioning of the organ in kidney, lung, adrenal, liver, bowel, cardiovascular system, and/or haemostatic system. Examples of organ damage include, but are not limited to, morphological/structural damage and/or damage to the functioning of the organ such as, for example accumulation of proteins (for example surfactant) or fluids due to pulmonary clearance impairment or damage to the pulmonary change mechanisms or alveolo-capillary membrane damage. The term “organ injury”, “organ damage” and “organ failure” may be used interchangeably. Normally, organ damage results in organ failure. By organ failure is meant a decrease in organ function compared to the mean, normal functioning of a corresponding organ in a person not having ALI or ARDS. The organ failure may be a minor decrease in function (e.g., 80-90% of normal) or it may be a major decrease in function (e.g., 10-20% of normal); the decrease may also be a complete failure of organ function. Organ failure includes, without limitation, decreased biological functioning (e.g., urine output), e.g., due to tissue necrosis, loss of glomeruli (kidney), fibrin deposition, haemorrhage, oedema, or inflammation. Organ damage includes, without limitation, tissue necrosis, loss of glomeruli (kidney), fibrin deposition, haemorrhage, oedema, or inflammation.

The term “lung damage” encompasses, but is not limited to, lung damage due to, for example, a congenital abnormality or an aquired abnormality such as that due to the on-set of an autoimmune condition, post-transplant lung rejection, infections resulting in an inflammatory response, changes in pressure/volume relationships in the lung, exposure of said mammal to a foreign agent (for example cigarette smoke or dust), a noxious or toxic agent (for example solvents or fumes) or is an undesirable side effect resulting from exposure to a therapeutic agent. Examples of lung damage include, but are not limited to, morphological/structural damage and/or damage to the functioning of the lung such as, for example accumulation of proteins (for example surfactant) or fluids due to pulmonary clearance impairment or damage to the pulmonary change mechanisms or alveolo-capillary membrane damage. The term “lung injury”, “lung damage” and “lung failure” may be used interchangeably.

Methods for testing organ function and efficiency, and suitable biochemical or clinical parameters for such testing, are well known to the skilled clinician.

Such markers, or biochemical parameters of organ function are, for example

| | |
|---|---|
| Respiration: | PaO2/FiO2 ratio |
| Coagulation: | Platelets |
| Liver: | Bilirubin |
| Cardiovascular: | Blood pressure and need for vasopressor treatment |
| Renal: | Creatinine and urine output |

Other clinical assessments could comprise ventilator free days, organ failure free days, vasopressor treatment free days, SOFA score and Lung Injury Score evaluation as well as vital signs.

Methods for testing for coagulophathy or inflammation are also well known to the skilled clinician. Such markers of coagulatory or inflammatory state are, for example, PTT, Fibrinogen depletion, elevation in TAT complexes, ATIII activity, IL-6, IL-8, and TNFR-1.

The term “chronic organ damage” encompasses, but are not limited to, the long-term damages that may result from having had ALI or ARDS. This residual impairment, in particular of pulmonary mechanics, may include, without restriction, mild restriction, obstruction, impairment of the diffusing capacity for carbon monoxide, or gas-exchange abnormalities with exercise, fibrosing alveolitis with persistent hypoxemia, increased alveolar dead space, and a further decrease in alveolar or pulmonary compliance. Pulmonary hypertension, owing to obliteration of the pulmonary-capillary bed, may be severe and lead to right ventricular failure.

In the present context, the term “treatment” includes treatment of established ALI, treatment of established ARDS, as well as preventing established ALI from developing into ARDS. Treatment includes the attenuation, elimination, minimization, alleviation or amelioration of symptoms or conditions associated with ALI or ARDS, including, but not limited to, the prevention of further damage and/or failure to organs already subject to some degree of organ failure and/or damage, as well as the prevention of development of damage and/or failure of additional organs not subject to organ failure and/or damage, at the time of administering modified FVII. Examples of such symptoms or conditions include, but are not limited to, morphological/structural damage and/or damage to the functioning of organs such as, but not limited to, lung, kidney, adrenal, liver, bowel, cardiovascular system, and/or haemostatic system. Examples of such symptoms or conditions include, but are not limited to, morphological/structural damage and/or damage to the functioning of the organs such as, for example, accumulation of proteins (for example surfactant) or fluids due to pulmonary clearance impairment or damage to the pulmonary exchange mechanisms or damage to the alveolo-capillary membrane, decreased urine output (kidney), tissue necrosis, loss of glomeruli (kidney), fibrin deposition, haemorrhage, oedema, or inflammation.

By “Attenuation” of organ failure or damage is meant an improvement in organ function as measured by at least one of these well known markers of function of said organs; when the organ failure or damage is attenuated the values of the selected markers are normalized compared to the values found in a human not having ALI or ARDS.

By “established” ALI or ARDS is meant that the patient have been assessed according to the above-mentioned four-point lung-injury scoring system as having ALI or ARDS (Bernard et al., Am.J.Respir.Crit.Care Med 149: 818-24, 1994), or that symptoms or conditions associated with ALI or ARDS have been observed in the patient.

Acute lung injury (ALI) may develop following exposure to a number of lung injury factors such as, but not limited to, aspiration of gastric contents, pneumonia, sepsis, massive transfusion, multiple trauma and pancreatitis. A smaller number of patients develop a more severe lung injury, referred to a adult or acute respiratory distress syndrome (ARDS) with a mortality of around 40-50%. ARDS may develop following exposure to a number of lung injury factors such as, but not limited to, aspiration of gastric contents, pneumonia, sepsis, massive transfusion, multiple trauma and pancreatitis.

In this context, the term “modified factor VII” is used interchangeably with “site-inactivated factor VIIa”, “active site-inactivated factor VIIa”, or “FVIIai”. Modified Factor VII, or FVIIai, can be in the form of the zymogen (i.e., a single-chain molecule) or can be cleaved at its activation site. Thus, “modified Factor VII” is meant to include modified Factor VII and modified Factor VIIa molecules that bind tissue factor and inhibit the activation of Factor IX to IXa and Factor X to Xa. Human FVIIa is disclosed, e.g., in U.S. Pat. No. 4,784,950 (wild-type factor VII). The Factor VII sequence has at least one amino acid modification, where the modification is selected so as to substantially reduce the ability of activated Factor VII to catalyze the activation of plasma Factors X or IX, and thus is capable of inhibiting clotting activity. The modified Factor VII has an active site modified by at least one amino acid substitution, and in its modified form is capable of binding tissue factor. The modified Factor VII compositions are typically in substantially pure form.

In preferred embodiments of human and bovine Factor VII, the active site residue Ser₃₄₄ is modified, replaced with Gly, Met, Thr, or more preferably, Ala. Such substitution could be made separately or in combination with substitution(s) at other sites in the catalytic triad, which includes His₁₉₃ and Asp₂₄₂.

Modified Factor VII may be encoded by a polynucleotide molecule comprising two operatively linked sequence coding regions encoding, respectively, a pre-pro peptide and a gla domain of a vitamin K-dependent plasma protein, and a gla domain-less Factor VII protein, wherein upon expression said polynucleotide encodes a modified Factor VII molecule which does not significantly activate plasma Factors X or IX, and is capable of binding tissue factor. The modified Factor VII molecule expressed by this polynucleotide is a biologically active anticoagulant, that is, it is capable of inhibiting the coagulation cascade and thus the formation of a fibrin deposit or clot. To express the modified Factor VII the polynucleotide molecule is transfected into mammalian cell lines, such as, for example, BHK, BHK 570 or 293 cell lines.

The catalytic activity of Factor VIIa can be inhibited by chemical derivatization of the catalytic center, or triad. Derivatization may be accomplished by reacting Factor VII with an irreversible inhibitor such as an organophosphor compound, a sulfonyl fluoride, a peptide halomethyl ketone or an azapeptide, or by acylation, for example. Preferred peptide halomethyl ketones include PPACK (D-Phe-Pro-Arg chloromethyl-ketone; (see U.S. Pat. No. 4,318,904, incorporated herein by reference), D-Phe-Phe-Arg and Phe-Phe-Arg chloromethylketone (FFR-cmk); and DEGRck (dansyl-Glu-Gly-Arg chloromethylketone).

The catalytic activity of Factor VIIa can also be inhibited by substituting, inserting or deleting amino acids. In preferred embodiments amino acid substitutions are made in the amino acid sequence of the Factor VII catalytic triad, defined herein as the regions which contain the amino acids which contribute to the Factor VIIa catalytic site. The substitutions, insertions or deletions in the catalytic triad are generally at or adjacent to the amino acids which form the catalytic site. In the human and bovine Factor VII proteins, the amino acids which form a catalytic “triad” are Ser₃₄₄, Asp₂₄₂, and His₁₉₃ (subscript numbering indicating position in the sequence). The catalytic sites in Factor VII from other mammalian species may be determined using presently available techniques including, among others, protein isolation and amino acid sequence analysis. Catalytic sites may also be determined by aligning a sequence with the sequence of other serine proteases, particularly chymotrypsin, whose active site has been previously determined (Sigler et al., J. Mol. Biol., 35:143-164 (1968), incorporated herein by reference), and therefrom determining from said alignment the analogous active site residues.

The amino acid substitutions, insertions or deletions are made so as to prevent or otherwise inhibit activation by the Factor VIIa of Factors X and/or IX. This can easily be determined by means of e.g., measuring the ability of Factor VIIa to produce of Factor Xa in a system comprising TF embedded in a lipid membrane and Factor X. (Persson et al., J. Biol. Chem. 272:19919-19924, 1997); or measuring Factor X hydrolysis in an aqueous system (see, “In vitro proteolytic assay” below). The Factor VII so modified should, however, also retain the ability to compete with authentic Factor VII and/or Factor VIIa for binding to tissue factor in the coagulation cascade. Such competition may readily be determined by means of, e.g., a clotting assay as described herein (e.g., as described in U.S. Pat. No. 5,997,864), or a competition binding assay using, e.g., a cell line having cell-surface tissue factor, such as the human bladder carcinoma cell line J82 (Sakai et al. J. Biol. Chem. 264: 9980-9988 (1989), incorporated by reference herein), or by measuring its physical binding to TF using an instrument based on surface plasmon resonance (e.g., Persson, FEBS Letts. 413:359-363, 1997)

The amino acids that form the catalytic site in Factor VII, such as Ser₃₄₄, Asp₂₄₂, and His₁₉₃ in human and bovine Factor VII, may either be substituted or deleted. Within the present invention, it is preferred to change only a single amino acid, thus minimizing the likelihood of increasing the antigenicity of the molecule or inhibiting its ability to bind tissue factor, however two or more amino acid changes (substitutions, additions or deletions) may be made and combinations of substitution(s), addition(s) and deletion(s) may also be made. In a preferred embodiment for human and bovine Factor VII, Ser₃₄₄ is preferably substituted with Ala, but Gly, Met, Thr or other amino acids can be substituted. It is preferred to replace Asp with Glu and to replace His with Lys or Arg. In general, substitutions are chosen to disrupt the tertiary protein structure as little as possible. The model of Dayhoff et al. (in Atlas of Protein Structure 1978, Nat'l Biomed. Res. Found., Washington, D.C.), incorporated herein by reference, may be used as a guide in selecting other amino acid substitutions. One may introduce residue alterations as described above in the catalytic site of appropriate Factor VII sequence of human, bovine or other species and test the resulting protein for a desired level of inhibition of catalytic activity and resulting anticoagulant activity as described herein. For the modified Factor VII the catalytic activity will be substantially inhibited, generally less than about 5% of the catalytic activity of wild-type Factor VII of the corresponding species, more preferably less than about 1% (e.g., as measured in the “in vitro proteolysis assay” below).

The modified Factor VII may be produced through the use of recombinant DNA techniques. In general, a cloned wild-type Factor VII DNA sequence is modified to encode the desired protein. This modified sequence is then inserted into an expression vector, which is in turn transformed or transfected into host cells. Higher eukaryotic cells, in particular cultured mammalian cells, are preferred as host cells. The complete nucleotide and amino acid sequences for human Factor VII are known. See U.S. Pat. No. 4,784,950, which is incorporated herein by reference, where the cloning and expression of recombinant human Factor VII is described. The bovine Factor VII sequence is described in Takeya et al., J. Biol. Chem. 263:14868-14872 (1988), which is incorporated by reference herein.

The amino acid sequence alterations may be accomplished by a variety of techniques. Modification of the DNA sequence may be by site-specific mutagenesis. Techniques for site-specific mutagenesis are well known in the art and are described by, for example, Zoller and Smith (DNA 3:479-488, 1984). Thus, using the nucleotide and amino acid sequences of Factor VII, one may introduce the alteration(s) of choice.

The Factor VII modified accordingly includes those proteins that have the amino-terminal portion (gla domain) substituted with a gla domain of one of the vitamin K-dependent plasma proteins Factor IX, Factor X, prothrombin, protein C, protein S or protein Z. The gla domains of the vitamin K-dependent plasma proteins are characterized by the presence of gamma-carboxy glutamic acid residues and are generally from about 30 to about 40 amino acids in length with C-termini corresponding to the positions of exon-intron boundaries in the respective genes. Methods for producing Factor VII with a heterologous gla domain are disclosed in U.S. Pat. No. 4,784,950, incorporated by reference herein.

DNA sequences for use in producing modified Factor VII will typically encode a pre-pro peptide at the amino-terminus of the Factor VII protein to obtain proper post-translational processing (e.g. gamma-carboxylation of glutamic acid residues) and secretion from the host cell. The pre-pro peptide may be that of Factor VII or another vitamin K-dependent plasma protein, such as Factor IX, Factor X, prothrombin, protein C or protein S. As will be appreciated by those skilled in the art, additional modifications can be made in the amino acid sequence of the modified Factor VII where those modifications do not significantly impair the ability of the protein to act as an anticoagulant. For example, the Factor VII modified in the catalytic triad can also be modified in the activation cleavage site to inhibit the conversion of zymogen Factor VII into its activated two-chain form, as generally described in U.S. Pat. No. 5,288,629, incorporated herein by reference.

Modified Factor VII may be purified by affinity chromatography on an anti-Factor VII antibody column. The use of calcium-dependent monoclonal antibodies, as described by Wakabayashi et al., J. Biol. Chem. 261:11097-11108, (1986) and Thim et al., Biochem. 27: 7785-7793, (1988), incorporated by reference herein, is particularly preferred. Additional purification may be achieved by conventional chemical purification means, such as high performance liquid chromatography. Other methods of purification, including barium citrate precipitation, are known in the art, and may be applied to the purification of the novel modified Factor VII described herein (see generally Scopes, R., Protein Purification, Springer-Verlag, N.Y., 1982). Substantially pure modified Factor VII of at least about 90 to 95% homogeneity is preferred, and 98 to 99% or more homogeneity most preferred, for pharmaceutical uses. Once purified, partially or to homogeneity as desired, the modified Factor VII may then be used therapeutically.

The modified Factor VII is cleaved at its activation site to convert it to its two-chain form. Activation may be carried out according to procedures known in the art, such as those disclosed by Osterud, et al., Biochemistry 11:2853-2857 (1972); Thomas, U.S. Pat. No. 4,456,591; Hedner and Kisiel, J. Clin. Invest. 71:1836-1841 (1983); or Kisiel and Fujikawa, Behring Inst. Mitt. 73:29-42 (1983), which are incorporated herein by reference. The resulting molecule is then formulated and administered as described below.

Administration and Dosing:

The pharmaceutical compositions for treatment of lung failure are intended for parenteral administration. Preferably, the pharmaceutical compositions are administered parenterally, i.e., intravenously, subcutaneously, intramuscularly, or pulmonary. The compositions for parenteral administration comprise a solution of the modified Factor VII molecules dissolved in an acceptable carrier, preferably an aqueous carrier. A variety of aqueous carriers may be used, e.g., water, buffered water, 0.4% saline, 0.3% glycine and the like. The modified Factor VII molecules can also be formulated into liposome preparations for delivery or targeting to sites of injury. Liposome preparations are generally described in, e.g., U.S. Pat. No. 4,837,028, U.S. Pat. No. 4,501,728, and U.S. Pat. No. 4,975,282, incorporated herein by reference. The compositions may be sterilized by conventional, well known sterilization techniques. The resulting aqueous solutions may be packaged for use or filtered under aseptic conditions and lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, etc. The concentration of modified Factor VII in these formulations can vary widely, i.e., from less than about 0.5%, usually at or at least about 1% to as much as 15 or 20% by weight and will be selected primarily by fluid volumes, viscosities, etc., in accordance with the particular mode of administration selected.

Thus, a typical pharmaceutical composition for intravenous infusion could be made up to contain 250 ml of sterile Ringer's solution, and 10 mg of modified Factor VII. Actual methods for preparing parenterally administrable compounds will be known or apparent to those skilled in the art and are described in more detail in for example, Remington's Pharmaceutical Science, 16th ed., Mack Publishing Company, Easton, Pa. (1982), which is incorporated herein by reference.

The compositions containing the modified Factor VII molecules are administered to a patient already suffering from a disease, as described above, in an amount sufficient to cure or at least partially arrest the disease and its complications. An amount adequate to accomplish this is defined as “therapeutically effective dose.” Amounts effective for this use will depend on the severity of the disease or injury and the weight and general state of the patient, but generally range from about 0.05 mg to 500 mg/day, more typically 1 mg to 200 mg/day, such as, for example, 1 mg to about 150 mg/day, 1 mg to about 125 mg/day, 1 mg to about 100 mg/day, 10 mg to about 175 mg/day, 10 mg to about 150 mg/day, or 10 mg to about 125 mg/day for a 70 kg patient as loading and maintenance doses.

It must be kept in mind that the materials of the present invention may generally be employed in serious disease or injury states, that is, life-threatening or potentially life threatening situations. In such cases, in view of the minimization of extraneous substances and general lack of immunogenicity of modified human Factor VII in humans, it is possible and may be felt desirable by the treating physician to administer substantial excesses of these modified Factor VII compositions.

The medicament can be administered by way of single or multiple administrations. For patients requiring daily maintenance levels, the modified Factor VII may be administered by repeated iv. injections or by continuous infusion using a portable pump system, for example. A pattern for administration of modified FVIIa in treatment of ARDS is, for example, a dose of about 1 mg/kg iv as loading dose followed by about 0.05 mg/kg/hr as maintenance dose (mg/kg designates mg modified factor VII per kg bodyweight of patient). Another patern is, for example, administering one or more doses of modified FVII per day (24 hours), e.g., 100 μg/kg×1, 100 μg/kg×2, 100 μg/kg×4, 200 μg/kg×1, 200 μg/kg×2, 200 μg/kg×4, 400 μg/kg×1, 400 μg/kg×2, 400 μg/kg×4, 800 μg/kg×1, or 800 μg/kg×2. This dosing can be administered for one or more days, e.g., (100 μg/kg×1 per day)×2 days, (100 μg/kg×2)×2 days, (100 μg/kg×4)×2 days, (200 μg/kg×1)×2 days, (200 μg/kg×2)×2 days, (200 μg/kg×4)×2 days, (400 μg/kg×1)×2 days, (400 μg/kg×2)×2 days, (400 μg/kg×4)×2 days, (800 μg/kg×1)×2 days, or (800 μg/kg×2)×2 days. The medicament is preferably administered as iv. Injections.

Modified FVII or another TF antagonist (e.g., anti-TF antibody) may also be administered in combination with activated Protein C (APC) or a fragment or variant thereof retaining APC's biological activity. In this case, a first amount of a Modified FVII or a TF antagonist and a second amount of APC or a biologically active variant or fragment thereof are administered, wherein the first and second amount together are effective in treatment of ALI or ARDS.

The composition may be in the form of a single preparation (single-dosage form) comprising both a preparation of modified FVII or another TF antagonist and a preparation of APC or a biologically active fragment or variant thereof in suitable concentrations. The composition may also be in the form of a kit-of-parts consisting of a first unit dosage form comprising a preparation of modified FVII or another TF antagonist and a second unit dosage form comprising a preparation of APC or a biologically active fragment or variant thereof. Either component may be administered first. Whenever a first or second or third, etc., unit dose is mentioned throughout this specification this does not indicate the preferred order of administration, but is merely done for convenience purposes. Preferably, both products are injected through the same intravenous access. The kit includes container means for containing the separate compositions such as a divided bottle or a divided foil packet. Typically the kit includes directions for the administration of the separate components. The kit form is particularly advantageous when the separate components are preferably administered in different dosage forms, are administered at different dosage intervals, or when titration of the individual components of the combination is desired by the prescribing physician.

The amount of modified FVII or another TF antagonist and the amount of APC or a biologically active fragment or variant thereof administered according to the present invention may vary from a ratio of between about 1:100 to about 100:1 (w/w). The ratio of modified FVII or another TF antagonist to APC or biologically active fragment or variant may thus be, e.g., about 1:100, or 1:90, or 1:80, or 1:70 or 1:60, or 1:50, or 1:40, or 1:30, or 1:20, or 1:10, or 1:5, or 1:2, or 1:1, or 2:1, or 5:1, or 10:1, or 20:1, or 30.1, or 40:1, or 50:1, or 60:1, or 70:1, or 80:1, or 90:1, or 100:1; or between about 1:90 to about 1:1, or between about 1:80 to about 1:2, or between about 1:70 to about 1:5, or between about 1:60 to about 1:10, or between about 1:50 to about 1:25, or between about 1:40 to about 1:30, or between about 90:1 to about 1:1, or between about 80:1 to about 2:1, or between about 70:1 to about 5:1, or between about 60:1 to about 10:1, or between about 50:1 to about 25:1, or between about 40:1 to about 30:1; or between about 10:1 to about 1:10, or between about 5:1 to about 1:5.

Modified FVII or another TF antagonist (e.g., anti-TF antibody) may also be administered in combination with TFPI or a fragment or variant thereof retaining TFPI's biological activity In this case, a first amount of a modified FVII or another TF antagonist and a second amount of TFPI or a biologically active variant or fragment thereof are administered, wherein the first and second amount together are effective in treatment of ALI or ARDS.

The composition may be in the form of a single preparation (single-dosage form) comprising both a preparation of modified FVII or another TF antagonist and a preparation of TFPI or a biologically active fragment or variant thereof in suitable concentrations. The composition may also be in the form of a kit-of-parts consisting of a first unit dosage form comprising a preparation of modified FVII or another TF antagonist and a second unit dosage form comprising a preparation of TFPI or a biologically active fragment or variant thereof. Either component may be administered first. Whenever a first or second or third, etc., unit dose is mentioned throughout this specification this does not indicate the preferred order of administration, but is merely done for convenience purposes. Preferably, both products are injected through the same intravenous access. The kit includes container means for containing the separate compositions such as a divided bottle or a divided foil packet. Typically the kit includes directions for the administration of the separate components. The kit form is particularly advantageous when the separate components are preferably administered in different dosage forms, are administered at different dosage intervals, or when titration of the individual components of the combination is desired by the prescribing physician.

The amount of modified FVII or another TF antagonist and the amount of TFPI or a biologically active fragment or variant thereof administered according to the present invention may vary from a ratio of between about 1:100 to about 100:1 (w/w). The ratio of modified FVII or another TF antagonist to TFPI or bilogically active variant or fragment thereof may thus be, e.g., about 1:100, or 1:90, or 1:80, or 1:70 or 1:60, or 1:50, or 1:40, or 1:30, or 1:20, or 1:10, or 1:5, or 1:2, or 1:1, or 2:1, or 5:1, or 10:1 or 20:1, or 30.1, or 40:1, or 50:1, or 60:1, or 70:1, or 80:1, or 90:1, or 100:1; or between about 1:90 to about 1:1, or between about 1:80 to about 1:2, or between about 1:70 to about 1:5, or between about 1:60 to about 1:10, or between about 1:50 to about 1:25, or between about 1:40 to about 1:30, or between about 90:1 to about 1:1, or between about 80:1 to about 2:1, or between about 70:1 to about 5:1, or between about 60:1 to about 10:1, or between about 50:1 to about 25:1, or between about 40:1 to about 30:1; or between about 10:1 to about 1:10, or between about 5:1 to about 1:5.

Modified FVII or another TF antagonist (e.g., anti-TF antibody) may also be administered in combination with a blood glucose lowering agent, e.g., insulin, preferably capable of maintaing blood glucose at or below 110 mg per deciliter patient plasma. In this case, a first amount of a modified FVII or another TF antagonist and a second amount of blood glucose-lowering agent, e.g., insulin or a biologically active variant or fragment thereof, are administered, wherein the first and second amount together are effective in treatment of ALI or ARDS.

The composition may be in the form of a single preparation (single-dosage form) comprising both a preparation of modified FVII or another TF antagonist and a preparation of blood glucose-lowering agent, e.g., insulin or a biologically active variant or fragment thereof, in suitable concentrations. The composition may also be in the form of a kit-of-parts consisting of a first unit dosage form comprising a preparation of modified FVII or another TF antagonist and a second unit dosage form comprising a preparation of blood glucose-lowering agent, e.g., insulin or a biologically active variant or fragment thereof. Either component may be administered first. Whenever a first or second or third, etc., unit dose is mentioned throughout this specification this does not indicate the preferred order of administration, but is merely done for convenience purposes. Preferably, both products are injected through the same intravenous access. The kit includes container means for containing the separate compositions such as a divided bottle or a divided foil packet. Typically the kit includes directions for the administration of the separate components. The kit form is particularly advantageous when the separate components are preferably administered in different dosage forms, are administered at different dosage intervals, or when titration of the individual components of the combination is desired by the prescribing physician.

The amount of modified FVII or another TF antagonist and the amount of blood glucose-lowering agent, e.g., insulin or a biologically active variant or fragment thereof, administered according to the present invention may vary from a ratio of between about 1:100 to about 100:1 (w/w). The ratio of factor VII to blood lowering agent may thus be, e.g., about 1:100, or 1:90, or 1:80, or 1:70 or 1:60, or 1:50, or 1:40, or 1:30, or 1:20, or 1:10, or 1:5, or 1:2, or 1:1, or 2:1, or 5:1, or 10:1, or 20:1, or 30.1, or 40:1, or 50:1, or 60:1, or 70:1, or 80:1, or 90:1, or 100:1; or between about 1:90 to about 1:1, or between about 1:80 to about 1:2, or between about 1:70 to about 1:5, or between about 1:60 to about 1:10, or between about 1:50 to about 1:25, or between about 1:40 to about 1:30, or between about 90:1 to about 1:1, or between about 80:1 to about 2:1, or between about 70:1 to about 5:1, or between about 60:1 to about 10:1, or between about 50:1 to about 25:1, or between about 40:1 to about 30:1; or between about 10:1 to about 1:10, or between about 5:1 to about 1:5.

Description of Experiments and Baboon Model:

Sepsis-induced tissue factor (TF) expression activates coagulation in the lung and leads to a pro-coagulant environment, which results in fibrin deposition and potentiates inflammation. Preventing initiation of coagulation at TF-Factor VIIa (FVIIa) complex blocks fibrin deposition and controls inflammation, thereby limiting acute lung injury (ALI) and other organ damage in sepsis. A baboon model of ALI was used where animals were primed with killed Escherichia coli (E.coli) (1×10⁹ CFU/kg), and lethal sepsis was induced 12 hours later by infusion of 1×10¹⁰ CFU/kg live E.coli. Animals in the treatment group were given a competitor inhibitor of TF, site-inactivated FVIIa (Modified FVII) intravenously at the time of infusion of live bacteria and monitored physilogically for another 36 hours. FVIIai dramatically protected gas exhange and lung compliance, prevented lung edema and pulmonary hypertension, and preserved renal function relative to vehicle (p<0.01) and decreased systemic pro-inflammatory cytokine responses, e.g. interleukin-6 (p<0.01). The protective effects of TF blockade in sepsis-induced ALI were confirmed using Tissue Factor Pathway Inhibitor (TFPI). The results show TF-FVIIa complex regulated organ injury in septic primates in part through selective stimulation of pro-inflammatory cytokine release and fibrin deposition.

Patients with gram-negative sepsis have a high incidence of acute respiratory distress syndrome (ARDS) and multiple organ failure (MOF). The lungs of these patients characteristically show fibrin deposition in alveolar and interstitial compartments although evidence that fibrin contributes to the pathogenesis of ARDS in sepsis is circumstantial. Strategies designed to treat sepsis by preventing disseminated intravascular coagulation (DIC) decrease mortality in humans and non-human primates with shock, but these studies have been limited by significant residual mortality, lack of organ specific analyses, and inability of the animal models to reproduce acute lung injury (ALI) that resembles ARDS. Because ARDS causes significant morbidity and mortality in septic patients, we used a non-human primate model of ARDS and MOF to investigate the contribution of tissue factor (TF) initiated coagulation and fibrin deposition to lung and systemic organ damage in sepsis.

When endotoxin or bacteria enter the circulation, extrinsic coagulation is rapidly activated and a procoagulant environment develops in the vascular space. This is dependent on TF and is associated with increases in inflammatory cytokines that mediate procoagulant effects of endotoxin. Similarly, procoagulant environments are found in the lungs of animals after endotoxin infusion or during experimental acute lung injury (ALI) and in bronchoalveolar lavage (BAL) of patients with ARDS. As in the systemic circulation, procoagulant activity in the lung is related to TF expression, suggesting that extravascular inflammation also activates the extrinsic pathway. Despite the association between procoagulant activity and lung injury, specific etiologic roles for TF and other coagulation factors have not been defined in the injury responses of the lung. Like TF, activated factors VII (FVIIa) and X (FXa), thrombin, and fibrin have specific effects on cell signalling that could alter vascular permeability, inflammatory cell migration, and surfactant dysfunction in the lung. The exact contribution of this complex cross-talk between coagulation and inflammation in the responses to sepsis is not known.

Blocking of coagulation during gram-negative sepsis prevents ALI and other organ damage by attenuating the coagulation-related inflammatory response. This was tested in an established baboon model of Escherichia coli (E.coli) sepsis where systemic inflammatory responses are pre-activated by a priming infusion of killed bacteria. After a second, lethal dose of bacteria, the animals develop hyperdynamic cardiovascular responses and pulmonary and renal failure similar to humans with ARDS. We blocked initiation of coagulation at the TF-FVIIa complex after the priming dose of bacteria using a site-inactivated FVIIa (FVIIai), which competitively inhibits FVIIa due to a five-fold higher affinity for TF than native FVIIa. The following study shows that coagulation blockade using FVIIai decreases systemic inflammation and fibrinogen depletion in sepsis syndrome and prevents injury to the lung and kidneys.

This is the first study to show specific improvements in end organ function after blocking initiation of coagulation in lethal sepsis. The findings establish an etiologic role for TF in sepsis-induced respiratory and renal failure and show that blockade of TF effectively preserves both pulmonary and renal function. This approach for evaluating therapeutic effects is powerful because the physiologic and histologic responses of primed baboons closely follow the responses to sepsis in humans. Previous animal studies using a variety of strategies to block coagulation in sepsis have reported better survival after either TF blockade or anticoagulation, but assessment of end organ injury has been complicated by the presence of severe septic shock. Priming preactivates inflammation and causes mild, self-limited alterations in pulmonary gas exchange, mechanics, and hemodynamics similar to experimental endotoxemia in humans. Subsequent overwhelming gram-negative sepsis results in progressive lung and renal injury, persistent elevation of inflammatory cytokines, and coagulophathy. The immune response in these animals is complex and certain therapies, e.g., mAb to leukocyte adhesion molecules, significantly worsen outcome in primed animals. In contrast, blockade of TF-FVIIa complex attenuates coagulophathy and fibrin deposition and prevents lung and renal injury after lethal E.coli infusion.

In the past, a primary goal of coagulation blockade in sepsis has been inhibition of fibrin deposition in the vascular compartment, but we have demonstrated that extravascular fibrin deposition during organ injury is also amenable to intervention. Fibrin provides the critical matrix for cell migration and collagen formation in tissue repair but may also stimulate inflammation. In the lungs, parenchymal accumulation of fibrin may contribute to inflammatory cell migration, surfactant dysfunction, and profibrotic processes. Although gas exchange and lung water were greatly improved in our study, residual fibrin was detected in the alveolar region and around small vessels in the lungs of FVIIai treated animals. This suggests that the strong protective effect of TF blockade were not entirely due to absence of fibrin and that key repair processes involving coagulation might remain intact during treatment with FVIIai.

FVIIai did prevent intraluminal fibrin clots in the lungs and kidneys after 36 hours of sepsis, which may have contributed to tissue protection. Intravascular fibrin deposition contributes to organ failure as a direct result of obstructive thrombus in small nutrient vessels and via enhancement of endothelial-leukocyte interactions. Although intravascular fibrin is likely to be important in some tissues and in certain clinical settings, for example when overwhelming shock and tissue hypoperfusion occur, extravascular TF expression by epithelial cells and tissue macrophages also initiates procoagulant, pro-inflammatory events. Both resident and infiltrating macrophages, as well as fixed cell populations, have been implicated as sources of TF in inflammatory lung and in renal disease suggesting coagulation is regulated differently in extravascular parenchyma.

TF is a Group II cytokine receptor that may regulate immune functions either directly or through generation of FXa, thrombin and fibrin, all of which exhibit cross-talk with inflammation. Each component has independent effects on the inflammatory response, and blocking initiation of TF has the advantage of curtailing inflammatory interactions at subsequent steps in the pathway. TF activated mitogen-activated protein kinase (MAPK) cytokine regulation relevant to the development of ALI. In particular, IL-6 has been associated with persistent inflammation and poor outcomes in ARDS. In vitro, FVIIai inhibits MAPK activation, demonstrating that catalytically active FVIIa is required for TF signalling via these pathways. Ligation of TF by FVIIa induces a number of immunoregulatory genes, including IL-1β, IL-8 and other chemokines, coagulation and growth factors, and collagenases. In our septic baboons, FVIIai decreased the plasma levels of IL-6, IL-8 and TNFR-1. This stems from decreased TF signalling or decreased downstream production of FXa and thrombin, which also induce pro-inflammatory cytokines. IL-6 and IL-8 further increase TF expression and TF blockade with FVIIai notably decreased sepsis-induced TF expression in the lung. Regulation of other important mediators of acute lung injury, e.g., VEGF, may require either generation of FXa by TF-FVIIa or the cytoplasmic tail of TF. Finally other data suggest that when TF is highly expressed it functions as a co-factor to present FVIIa to other transmembrane proteins that initiate signalling events. If such interactions are important when TF is highly over-expressed as in sepsis, direct targeting of FVIIa has an advantage over other inventions that inhibit TF.

In earlier studies of animals with fulminate sepsis, three experimental agents, TFPI, anti-TF mAb, and DEGR-FVIIa, have been targeted at TF-initiated coagulation. These agents improved survival, however, natural inhibitors of proteases distal to the TF-FVIIa complex, including activated Protein C (APC) and antithrombin III (AT III), have also shown survival efficacy. Because these strategies have all been tested in previously unchallenged animals that develop rapidly progressive shock, it is possible that coagulation activity contributes to mortality in shock downstream from the TF-FVIIa complex. Like the anti-TF agents, their impact has not been studied for ALI and MOF.

In the above studies, decreases in serum IL-6 and IL-8 were observed and considered as a mechanism for improved survival. Critical effects for these mediators have been difficult to localize and do not consistently link coagulation and cytokines with survival. AT III, which inhibits coagulation at FXa and thrombin, decreased mortality, coagulophathy and IL-6 production, however, these results were not duplicated in human trials. In contrast, DEGF-FVIIa attenuated coagulophathy and IL-6 production but had variable effects on survival that did not correlate with cytokine levels. Also, inactivated FXa attenuated coagulophathy but did not improved survival in acute septic shock. The effects of coagulation blockade in those studies were not correlated with physiologic endpoints of organ function. In humans TF blockade with TFPI did not affect IL-6 levels with low dose endotoxin infusion, although it did prevent activation of coagulation. Together these studies imply different thresholds for inflammatory and clotting functions of coagulation proteases in primates, especially as the inflammatory challenge progresses.

In our animals FVIIai abrogated lung inflammation without altogether blocking coagulation. The novel observation offers a promise to septic patients where bleeding is a concern. Although FVIIai binds TF effectively, it blocks coagulation incompletely in vitro. Thus greater activation of TF-FVIIa may be required for inflammation than coagulation and at the dose of FVIIai used in this study no serious bleeding was seen. In addition, the effect of the drug on coagulation can be reversed with human recombinant FVIIa if bleeding does occur.

In summary, we have shown that blockade of coagulation at the TF-FVIIa complex prevents lung and renal injury during E.coli sepsis in non-human primates. Other tissues were protected to varying degrees, suggesting TF-based contributions to injury in sepsis are different among organs. As in critically ill humans with ARDS, we tested this strategy in the presence of persistent inflammation, where prolonged cytokine expression may have critical implications for functional outcome. Previous strategies for septic shock based on different aspects of coagulation have had varying clinical success.

This likely reflects both the heterogenous injury of sepsis and interactions among different coagulation proteases with respect to inflammation. Our data suggest agents that act proximally in the coagulation cascade will have a greater positive impact on pulmonary and renal injury in sepsis.

The following examples are offered by way of illustration, not by way of limitation.

### EXAMPLES

### Example 1

Biological Activity of FVII

The activity of factor VIIa or factor VIIa variants may be measured using a physiological substrate such as factor X, suitably at a concentration of 100-1000 nM, where the factor Xa generated is measured after the addition of a suitable chromogenic substrate (eg. S-2765). In addition, the activity assay may be run at physiological temperature.

“In Vitro Proteolysis Assay”

Native (wild-type) Factor VIIa and Factor VIIa variant (both hereafter referred to as “Factor VIIa”) are assayed in parallel to directly compare their specific activities. The assay is carried out in a microtiter plate (MaxiSorp, Nunc, Denmark). Factor VIIa (10 nM) and Factor X (0.8 microM) in 100 microL 50 mM Hepes, pH 7.4, containing 0.1 M NaCl, 5 mM CaCl2 and 1 mg/ml bovine serum albumin, are incubated for 15 min. Factor X cleavage is then stopped by the addition of 50 microL 50 mM Hepes, pH 7.4, containing 0.1 M NaCl, 20 mM EDTA and 1 mg/ml bovine serum albumin. The amount of Factor Xa generated is measured by addition of the chromogenic substrate Z-D-Arg-Gly-Arg-p-nitroanilide (S-2765, Chromogenix, Sweden), final concentration 0.5 mM. The absorbance at 405 nm is measured continuously in a SpectraMax™ 340 plate reader (Molecular Devices, USA). The absorbance developed during 10 minutes, after subtraction of the absorbance in a blank well containing no FVIIa, is used to calculate the ratio between the proteolytic activities of variant and wild-type Factor VIIa:

Ratio=(A405 nm Factor VIIa variant)/(A405 nm Factor VIIa wild-type).

Based thereon, factor VIIa variants with an activity substantially lower than native factor VIIa may be identified, such as, for example, variants where the ratio between the activity of the variant and the activity of native factor VII (wild-type FVII) is below 5%, or 1%, or lower.

### Example 2

Blockade of Extrinsic Coagulation Decreases Lung Injury in Baboons with Established Gram-Negative Sepsis

It has been demonstrated that blockade of initiation of coagulation with active site-inactivated VIIa (ASIS) at the time of live bacteria infusion attenuated sepsis-associated acute lung injury (ALI) and renal failure in baboons. We have shown that established E. coli sepsis also respond to treatment with ASIS with decreased ALI and renal failure.

Adult male baboons received an infusion of 1×10⁹/kg heat-killed E. coli 12 hours prior to intravenous live E. coli 1×10¹⁰/kg. Animals were mechanically ventilated for 48 hours and supported with fluids to maintain a PCWP (pulmonary capillary wedge pressure) of 8-12 mmHg. Six animals were treated one hour after live bacterial infusion with ASIS (1 mg/kg iv followed by 50 μg/kg/hr). Six animals served as sepsis controls. Values shown as mean±SE.

ASIS prevented plasma fibrinogen depletion, consistent with therapeutic blockade of the extrinsic pathway. Sepsis induced neutropenia and thrombocytopenia were unaffected. After 48 hours, treated animals had preserved gas exchange (ΔAaDO2, mmHg: C=25.4±3.9, ASIS=14.4±5.2) with decreased lung wet/dry weights (C=6.9±0.8, ASIS=5.0±0.2). Lung histology showed decreased inflammation in the ASIS-treated septic animals. In septic animals treated with ASIS, urine output was higher (UOP, ml/kg/hr C=5.7±1, ASIS=12.3±1.7, p≦0.01) and metabolic acidosis was attenuated (ΔHCO₃—, meq/dl: C=−4.3±2.9, ASIS=+3±1.1, p≦0.05). Kidneys from ASIS-treated animals showed preserved tubular architecture compared to sepsis controls. Drug infusion was well tolerated without bleeding complications. The results show that inhibiting initiation of extrinsic coagulation protects against acute lung and renal in established sepsis.

| Group | ΔAaDO2 | Wet/dry | UOP | ΔHCO₃₋ |
|---|---|---|---|---|
| Sepsis control | 25.4 ± 3.9 | 6.9 ± 0.8 | 5.7 ± 1 | −4.3 ± 2.9 |
| ASIS | 14.4 ± 5.2 | 5.0 ± 0.2 | 12.3 ± 1.7 | +3 ± 1.1 |

ASIS is D-Phe-Phe-Arg-FVIIa.

### EXAMPLE 3

Tissue Factor Blockade in Experimental Acute Lung Injury

We studied blockade of TF-initiated coagulation in baboons with ALI from E.coli sepsis. Active site inactivated FVII (ASIS) blocked extrinsic coagulation and decreased systemic cytokine responses, including interleukin (IL)-6, IL-8 and tumour necrosis factor receptor-1. It also attenuated sepsis-related injury in the lung, kidney and other tissues. Measurements of plasma fibrinogen and thrombin-anti-thrombin III (TAT) complexes confirmed a decrease in intravascular activation of coagulation after treatment with ASIS.

In untreated septic animals, fibrin deposition was prominent in the lung and other tissues in both intra- and extra-vascular compartments. This was decreased but not eliminated in septic animals treated with ASIS, suggesting that protective effects of TF-blockade were not solely due to decreased generation of fibrin. TF blockade with ASIS also decreased inflammatory changes in the lung, including neutrophil infiltration, and decreased oedema and haemorrhage. Blockade of coagulation and attenuation of fibrin deposition by ASIS improved lung function by preserving gas exchange and compliance, decreased pulmonary hypertension, and improved renal function. Two septic baboons treated with TFPI also showed improvements in gas exchange and lung compliance although to a lesser extent than those treated with ASIS. These results show that TF-FVIIa complex is an important regulatory site for the pathological responses to sepsis.

One possible protective mechanism of coagulation blockade in sepsis is attenuation of pro-inflammatory cytokine production. The possibility that cross-talk between coagulation and inflammation is a key component of dysregulated inflammation has implications for the extent of end organ damage. In the lungs, TF expressed in alveolar and intestinal spaces by lung epithelial cells and macrophages may initiate procoagulant, pro-inflammatory events in sepsis, that when modified by TF blockade leads to improvements in pulmonary function.

ASIS is D-Phe-Phe-Arg-FVIIa.

### EXAMPLE 4

Methods

Animal preparation. Adult male baboons (Papio cyanocephalus) weighing 14 to 20 kg were quarantined for a minimum of four weeks, and determined to be tuberculosis-free prior to use. Animals were handled in accordance with AAALAC guidelines, and the experimental protocol was approved by the Duke University Institutional Animal Care and Use Commitee. They were divided randomly into treatment and sepsis control groups (n=6 each). Treated animals received active site-inactivated FVIIa (FVIIai, Novo Nordisk, Copenhagen) 1 mg/kg intravenously (iv.) at time t=12 h, immediate prior to the infusion of live bacteria, followed by 50 mcg/kg/h iv. Untreated animals received iv. Infusion of vehicle only. The drug is derived from human recombinant FVIIa, where the active site has been blocked by incorporation of a small peptide (D-Phe-L-Phe-L-Arg chloromethyl ketone), and the dose was selected on the basis of safety studies in human patients. The modification blocks proteolytic activity and enhances TF affinity five-fold. To confirm findings with an independent inhibitor of TF, two additional baboons were treated with the same protocol with tissue factor pathway inhibitor (TFPI, gift of Abla Creasy, Chiron, Emeryville, Calif.) at the same dose.

After an overnight fast each animal was sedated with intramuscular ketamine (20-25 mg/kg) and intubated. Heavy sedation was maintained with ketamine (3-10 mg/kg/h) and diazepam (0.4-0.8 mg/kg every 2 hours). Animals were ventilated with a volume-cycled ventilator and paralyzed intermittently with pancuronium (4 mg intraveneously) before respiratory measurements. The FiO₂ was 0.21, tidal volume 12 mg/kg, positive end-expiratory pressure 2.5 cm H₂O, and a rate adjusted to maintain an arterial PCO₂ of 40 mm Hg. An indwelling artrial line and a pulmonary artery cathetet were placed via femoral cut down for hemodynamic monitoring. Detailed descriptions of the model have been published (e.g., Welty-Wolf et al., Am J Resp Crit Care Med 1998; 158: 610-619).

All animals received approximately 10⁹ CFU/kg heat-killed E.coli as a 60 min infusion at t=o h, 12 h before live E.coli. Sepsis was induced at t=12 h by infusing 10¹⁰ CFU/kg of live E.coli in a volume of 50 ml over 60 min. Gentamicin (3 mg/kg iv.) and Ceftazidime (1 gm iv.) were administered 60 min after completion of the live E.coli infusion. Fluids were given as needed to maintain pulmonary capillary wedge pressure (PCWP) at 8-12 mm Hg and to support blood pressure. Dopamine was used for hypotension when mean arterial pressure (MAP) fell below 65 mm Hg despite fluids. After 48 h (36 h after the live bactria infusion) animals were deeply anesthetized and killed by KCl injection. Predefined termination criteria included refractory hypotension (MAP less than 60 mm Hg), hypoxemia (need for FiO₂ greater than 40%), or refractory metabolic acidosis (pH<7.10 with normal PaCO₂).

Hemodynamic monitoring. Physiologic parameters including heart rate (HR), temperature, arterial blood pressure, pulmonary artery pressure, ventilator parameters, and fluid intake were recorded every hour. Measurements were obtained every six hours of cardiac output (CO) by thermodilution, central venous pressure (CVP), PCWP, arterial and mixed venous blood gasses, oxygen saturation, oxygen content and hemoglobin (Hgb) as reported (e.g., Welty-Wolf et al., Am J Resp Crit Care Med 1998; 158: 610-619). Urinary catheter output was measured every six hours and fluid balance calculated as total iv. Intake minus urine output.

Preparation of E.coli. E.coli (American Type Culture Collection, Rockville, Md.; serotype 086a:K61) was prepared as described (REFS 7-10) and adjusted to give a final dose of 1×10¹⁰ CFU/kg for each baboon (LD₁₀₀). Heat-killed E.coli were prepared by heating tubes of bacteria in a water bath at 65° C. for at least 30 minutes. The number of organisms and efficacy of heat killing were confirmed by colony counting using pour plates.

Measurements on whole blood, plasma, and serum. Blood samples were drawn at 0, 12, 13, 18, 24, 36, and 48 h. Complete blood counts were performed on whole blood (Sysmex-1000 Hemocytometer, Sysmex, Inc., Long Grove, Ill.). Plasma (from citrated blood) and serum were separated and stored at −80° C. Fibrinogen was measured using an ST4 mechanical coagulation analyzed (Diagnostiga Stago, Parsippany, N.J.). Prothrombin time (PT) and activated partial thromboplastin time (aPTT) were measured in duplicate, and antithrombin III (AT III) activity was measured on an MDA coagulation analyzer (Organon Teknika; Durham, N.C.) with a chromogenic assay and expressed as % of the kit standard. ELISA was used to measure plasma thrombin-antithrombin (TAT) complexes (Dade Behring, Deerfield, Ill.) and FVIIai levels in plasma and BAL (Novo Nordisk, Copenhagen). Serum samples were assayed for interleukin 1β (IL-1β, IL-6, IL-8, and TNF receptor-1 (TNFR-1) using ELISA kits (R and D Systems, Inc., Minneapolis, Minn.). Blood urea nitrogen (BUN) and creatinine were measured with standard clinical techniques.

Tissue Collection and Preparation. After the experiments the thorax was opened, the left mainstem bronchus ligated, and the left lung removed. BAL was performed on the left upper lobe with 240 ml 0.9% saline. Samples of lung tissue from the left lower lobe were manually inflated and immersed in 4% paraformaldehyde for light microscopy and immunohistochemistry. Four samples were taken at random from the remainder of the left lung for wet/dry weight determination taking care to avoid large vascular and bronchial structures. Additional samples from lung, kidney, liver, small bowel, heart, and adrenal were flash frozed in liquid nitrogen and stored at −80° C. for Western blotting and biochemical studies. The entire right lung was inflation-fixed for 15 min at 30 cm fixative pressure with 2% glutaraldehyde in 0.85 M Na cacodylate buffer (pH 7.4). Additional tissue from kidney, liver, small bowel, heart, and adrenal was fixed by immersion in 4% paraformaldehyde. Four samples of small bowel were selected randomly for wet/dry weight determination.

Biochemical Measurements: myeloperoxidase (MPO) activity and protein concentration of lung homogenates and protein and lactate dehydrogenase (LDH) concentrations of BAL fluid were measured as described (e.g., Carraway et al., AM J Resp Crit Care 1998, 157: 938-949). MPO activity was expressed as a change in absorbance/min/g wet weight tissue. LDH values were expressed in units of activity per liter (U/L).

Western Blotting. Lung samples were homogenized in cold lysis buffer (150 mM NaCl, 50 mM Tris, pH 7.6 1% SDS, 3% Nonidet P-40, 5 mM EDTA, 1 mM MgCl₂, 2 mM 1,3-dichloroisocoumarin, 2 mM 1,10-phenanthroline, and 0.4 mM E-64) and centrifuged at 15,000×g for 10 min. The supernatants were mixed with Laemmli buffer and frozed at −80° C. electrophoresis was done under reducing conditions using 12% polyacrylamide gels. Lanes were loaded with equivalent amounts of protein and electrophoresis was performed on a Hoefer minigel system (Hoefer Scientific Instruments, San Francisco, Calif.). After transfer, blots were probed for TF expression using anti-TF mAb (mouse anti human, American Diagnostica, Greenwich, Conn.) and HRP-conjugated secondary Ab (goat anti.mouse, Transduction Laboratories, Lexington, Ky.). Signals were developed ECL detection and blots were densitized using commercially available software (Quantity One, BioRad, Hercules, Calif.).

Histology and Immunochemistry. Paraformaldehyde fixed tissues were embedded in paraffin, sectioned and stained with hematoxylin and eosin (H&E), and examined by light microscopy. Immunolocalization for fibrin was performed using a mAb (anti-human fibrinogen β-chain, American Diagnostica, Greenwich, Conn.) on paraffin sections of lung, kidney, adrenal, and small bowel. This Ab reacts strongly with fibrin and weakly with fibrinogen. Sections (5 microns) were deparaffinized in xylene, rehydrated in graded alcohol, and washed prior to incubating overnight at 4° C. with anti-fibrin Ab. Sections were then washed and incubated with biotinylated secondary Ab and the signal developed with peroxidase-conjugated avidin and aminobenzidine. Negative controls were processed as above except primary incubations were done with non-immune mouse serum (Jackson Laboratories, Bar Harbor, Me.).

Statistics. Data were entered into a computer spreadsheet and analyzed using commercially available software (StatView, Calabasas, Calif.). Physiologic data and data from serial blood draws were analyzed by two-factor ANOVA. Biochemical data from BAL and tissues obtained at the end of the experiments were analyzed using unpaired t-tests. Mean±sem and p values are provided in the figures and table; p<0.05 was considered significant and trends are noted for p<0.10.

Results

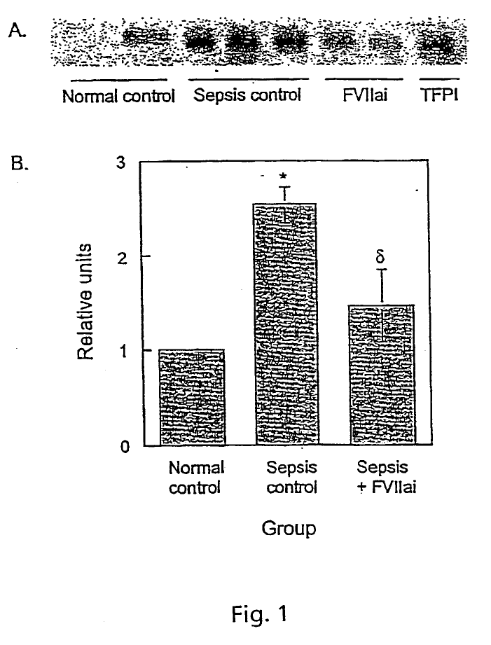

Both coagulation and inflammation were activated by dead bacteria before infusion of a lethal dose of live E.coli. Just prior to administration of live E.coli, the animals had a mild coagulopathy with increases in TAT complexes and aPTT, decreased platelets, and increased fibrinogen consistent with an acute phase response. The inflammatory mediators IL-6, IL-8, and TNFR-1 were increased 2-10 fold. Infusion of live bacteria in these animals caused extensive lung injury, renal insufficiency, and damage to other vital organs including liver, bowel, and adrenals. Intravenous administration of FVIIai as a constant infusion effectively blocked further activation of coagulation and inflammation, prevented organ injury, and diminished both intra- and extravascular fibrin deposition. The effect on tissue deposition of fibrin was most prominent in the lung and kidney, where FVIIai treated animals showed remarkable improvements in gas exchange and renal function compared to vehicle treated septic controls. Untreated sepsis control animals had strong up-regulation of TF in the lungs that was prevented with FVIIai (p<0.05, FIG. 1). Drug levels were measured in plasma and BAL, and showed penetration of FVIIai into the alveolar compartment, where levels in BAL fluid were 194.2±34.7 ng/mg protein at the end of the experiments. Plasma levels are shown in table 1. Analysis of the pulmonary and renal protection by FVIIai treatment in these animals is provided below.

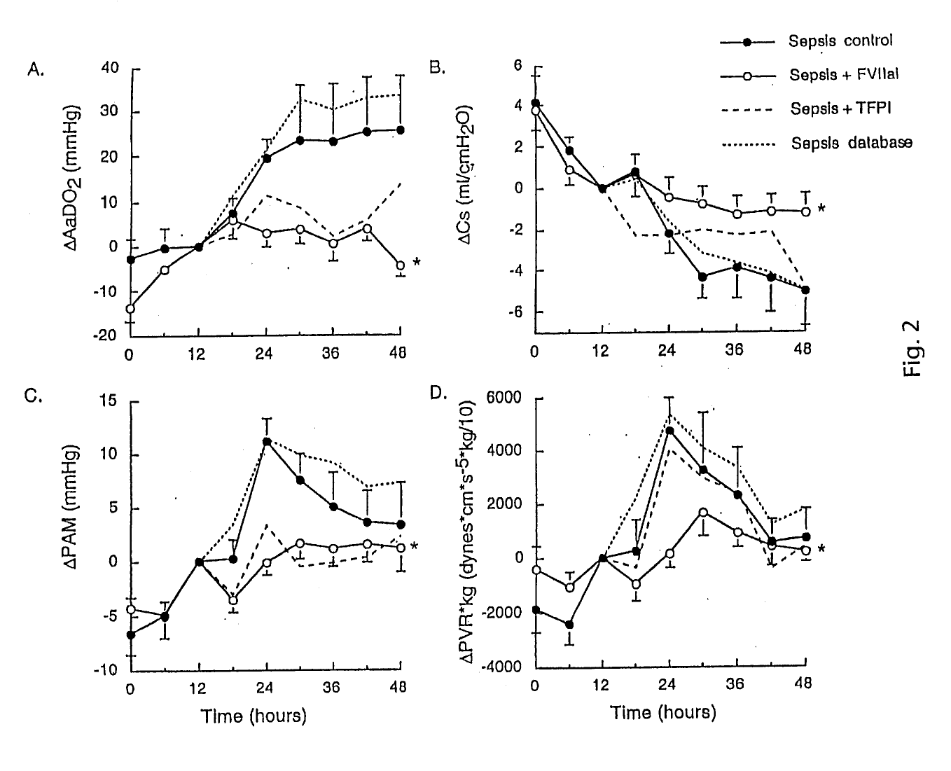

Acute lung injury in sepsis. FVIIai treatment prevented sepsis-induced hypoxemia, pulmonary hypertension, and loss of pulmonary system compliance. These physiologic data are shown in FIG. 2, plotted as change from t=12 h to show the drug effect. Historical data from earlier untreated animals (n=11) and two septic animals treated with TFPI are shown as broken lines on the graphs for comparison only (data not included in statistical analyses). Alveolar arterial oxygen gradient (AaDO₂) increased in both groups after infusion of killed bacteria and progressively worsened in the sepsis control group after the onset of live bacterial sepsis at t=12 h. One animal in the sepsis control required supplemental oxygen. Treatment with FVIIai prevented deterioration in gas exchange during sepsis (p<0.0001), and the final AaDO₂ actually improved in those animals compared to 12 h. Sepsis-induced increases in mean pulmonary artery pressure (PAM) and pulmonary vascular resistance kg (PVR*kg) were attenuated by FVIIai (p<0.001 and p<0.02 vs. untreated sepsis controls). FVIIai also prevented the loss of pulmonary system compliance seen in sepsis control animals (p<0.001). Dead space increased similarly and both groups required a 30-35% increase in minute ventilation (V_{E}) during the experiment (table 1). The PaCO₂ was controlled at 40 mm Hg in both groups (p=NS for both V_{E} and PaCO₂).

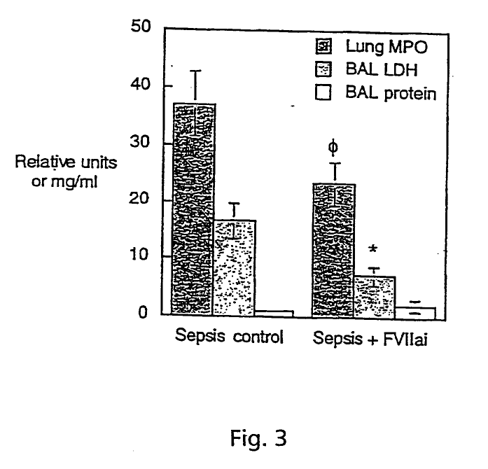

At post-mortem, the lungs of animals treated with FVIIai appeared normal, similar to lungs from uninjured ventilated animals. In contrast, the lungs from sepsis control animals were dense and hemorrhagic. Quantitative measures of lung wet/dry weight, neutrophils (PMN) accumulation, and lavage LDH were all improved in the treated group (FIG. 3). Lung wet/dry weights were 5.81±0.19 in septic controls compared to 5.05±0.09 in FVIIai treated animals (p<0.01, normal reference range is 4.6-5.0). BAL LDH decreased almost 60% (p<0.01) and lung MPO activity was decreased over 40% (p=0.07). BAL protein was not significantly different between the two groups.

Lung histology showed marked protection in septic animals treated with FVIIai. Representative sections of the lungs were stained with anti-fibrin antibody. The lungs of sepsis control animals has thickened alveolar septae, patchy alveolar edema and hemorrhage, and intra-alveolar inflammatory cell infiltration with macrophages and PMNs. Anti-fibrin staining showed extensive difuse fibrin deposition along the septae, on intra-alveolar inflammatory cells, and in alveolar fluid. Some small vessels in the lungs contained fibrin clots. Lungs of treated animals had normal alveolar septal architecture, minimal alveolar PMN infiltration, and no alveolar edema. In these animals, septal staining for fibrin was heterogeneous and less extensive that in sepsis controls. In the treated animals, fibrin staining was frequently limited to areas immediately surrounding small vessels, however, intravascular fibrin clots were not apparent. alveolar macrophages and intravascular monocytes stained focally.

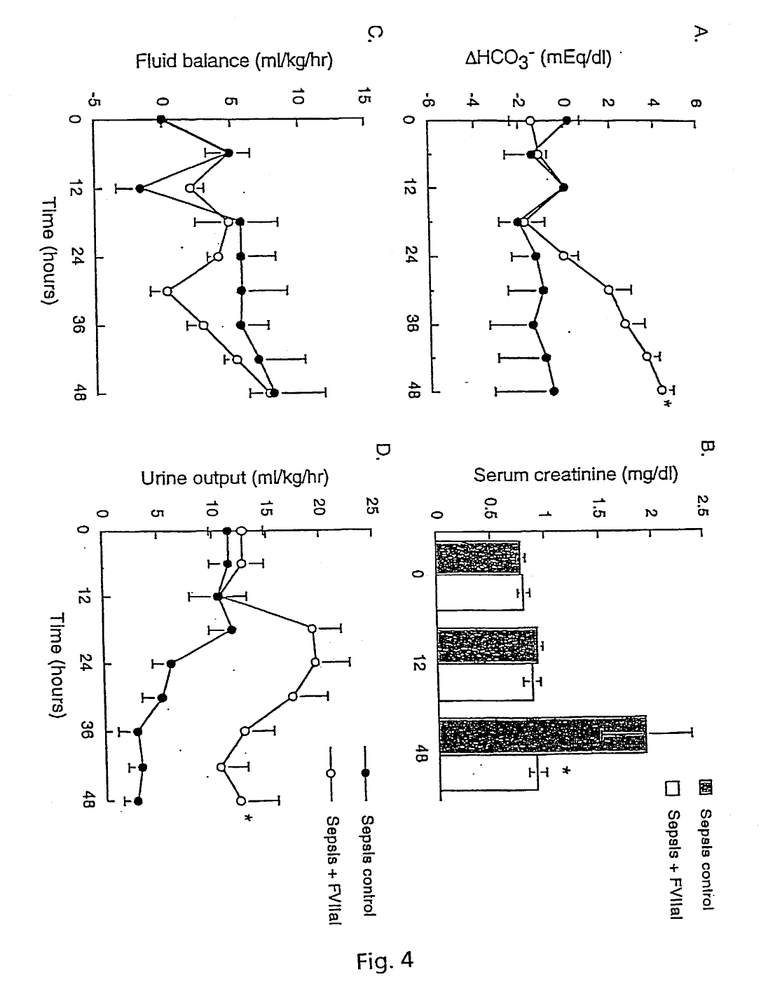

Renal and other organ damage in sepsis. FVIIai also prevented renal failure in sepsis (FIG. 4). Serum creatinine doubled in the sepsis control group but remained normal in the treatment group (p=0.05). In untreated animals, there was a corresponding decrease in urine output after infusion of live E.coli. In contrast, urine output was maintained or increased in the treatment group (p<0.0001). This was not due to differences in resuscitation because fluid balance (FIG. 4) and systemic hemodynamics (table 1) were similar in the two groups. Blood pH and serum [HCO₃⁻] were lower in untreated animals (p<0.001 and p<0.1 respectively, FIG. 4).

Kidneys from untreated animals were swollen and hemorrhagic at post mortem but appeared normal in FVIIai treated animals. H&E stained sections of the kidneys of untreated animals had patchy areas of acute tubular necrosis (ATN) and loss of glomeruli. The kidneys of treated animals, except for a few small foci of ATN, showed normal renal architecture. Immunostatining showed fibrin deposition in glomeruli of sepsis control animals with obliteration of capillary structure. Tubular epithelium also stained, and some tubules contained amorphous material that was also positive for fibrin. Vessels occluded by fibrin clot were readily identified. In the treated animals, glomerular fibrin deposition was absent and minimal tubular epithelial staining was seen in only a few animals.

The appearance of the adrenals, liver, and small bowel was also normal in the FVIIai treated animals. In contrast, the adrenals from untreated animals were swollen and hemorrhagic and small bowel was grossly edematous. Small bowel wet/dry weights were higher in untreated animals, but high variability in the bowel injury did not permit a statistical difference to be achieved between the groups (6.36±0.51 in treated vs. 8.30±1.13 in untreated animals, p=0.15). In contrast to the decreased fibrin staining in the lungs and kidneys, focal fibrin deposition was seen an adrenals and small bowel in both treated and untreated septic animals. Despite this adrenal cortical congestion and hemorrhage and small bowel hemorrhage and edema were diminished in septic animals treated with FVIIai. There was no statistical significant effect of FVIIai on PMN content in organs other than the lung. MPO activity in kidney, liver, and small bowel was variable in control animals and differences were not statistically significant between the two groups.

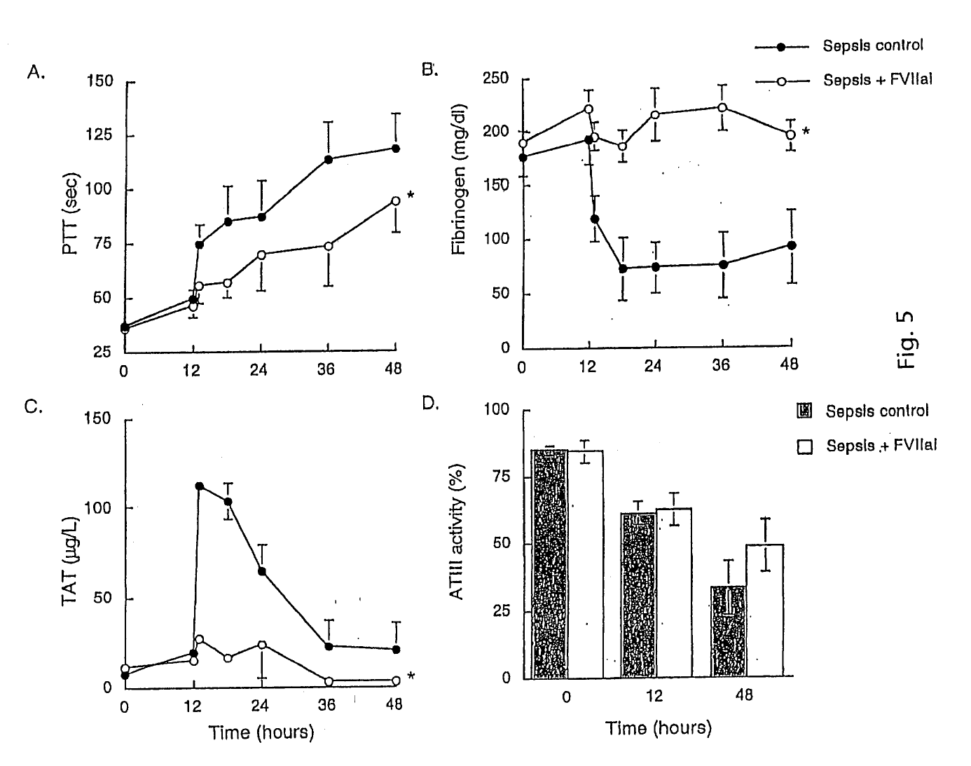

Sepsis-induced coagulopathy. Intravascular activation of coagulation was decreased in septic animals treated with FVIIai compared to controls (FIG. 5). Initial values for coagulation parameters were within the normal range for this species. Drug treatment prevented plasma fibrinogen depletion as expected with therapeutic blockade of coagulation (p<0.0001). TAT complexes increased after live E coli in sepsis controls, peaking at 13-18 h, and then declined as AT III activity levels decreased. The increase in TAT complexes was attenuated in treated animals (p<0.0001), however the decrease in AT III activity was not statistically different. Although TAT levels decreased late in the experiment in untreated septic animals, coagulation was ongoing in those baboons. The aPTT increased progressively in both groups but was higher in untreated animals (p<0.01). PT was higher in the treatment group due to drug effect on the assay, between 53 and 67 s for the duration of drug infusion (p<0.0001). In the untreated group PT increased progressively from 17.8±0.4 at 12 h (before live E.coli were infused) to 25.5±3.6 at the end of the experiment.

Both groups of animals developed neutropenia, thrombocytopenia and anemia after infusion of live E.coli (see table 1). WBC reached a nadir of approximately 1,500 (×10³/μl) in both groups one our after the infusion (t=13 h) and progressively increased to near baseline levels by the end of the experiment (9,400±1,800 in treated vs. 13,000±3,900 in untreated animals, p=0.08). all animals were thrombocytopenic by 12 h after the infusion of live E.coli (t=24 h) and mean platelet counts were 30,000 or less in both groups at the end of the experiment. Hgb decreased similarly in both groups without evidence of significant hemorrhage in either (table 1).

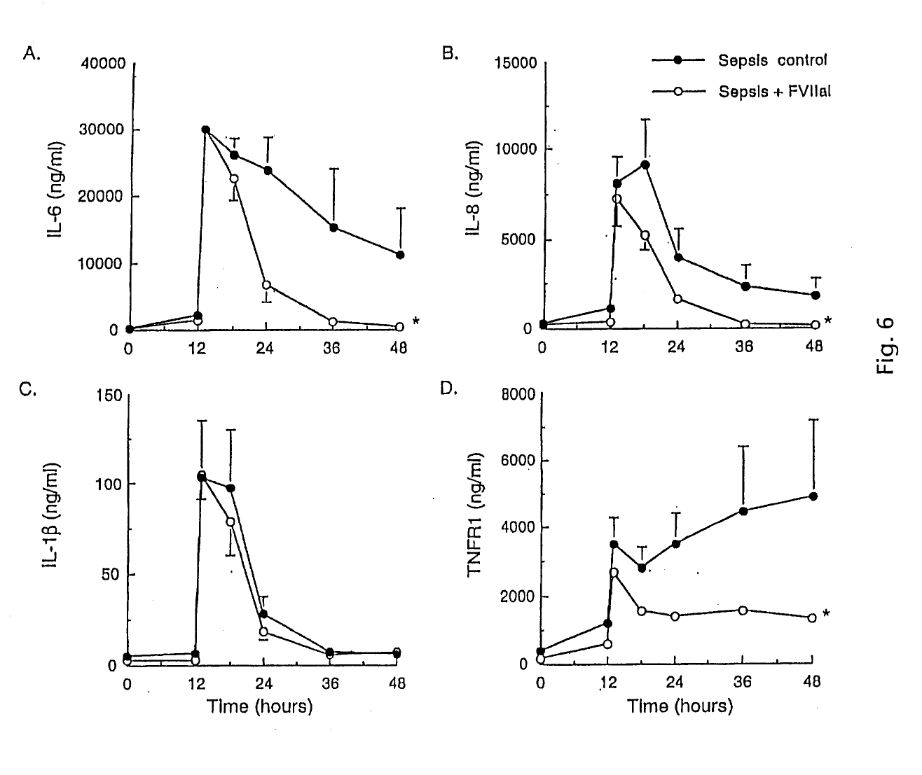

Pro-inflammatory cytokine levels. Elevations of inflammatory cytokines were attenuated by treatment with FVIIai (FIG. 6). Circulating levels of IL-1β, IL-6, IL-8, and TNFR-1 rose sharply after infusion of live E.coli in both treated and untreated animals. Peak IL-6 levels were not different between the two groups, but IL-6 declined more rapisly in FVIIai treated animals (p<0.001) and returned to levels found in naive animals. Likewise, IL-8 and TNFR-1 levels were attenuated compared to controls (p<0.01 and p<0.001). There was no difference in IL-8 levels between the two groups.

Systemic hemodynamic parameters. Hemodynamic measurements, including HR, MAP, PCWP, CO/kg, and systemic vascular resistance*kg (SVR*kg), were not altered by treatment with FVIIai (table 1). Hypotension responded to IV fluids in both groups; one animal in the treatment group required low dose dopamine briefly after live bacteria were infused. Ten of the 12 animals survived until the scheduled termination point of the protocol. One sepsis control animal died at 30 h (18 h after live bacteria infusion) from ALI, with refractory hypoxemia and respiratory acidosis, and one animal in the FVIIai treatment group died 3 h before the end of the study from a complication of endotracheal intubation. Two animals in each group developed self-limited hematuria during the experiment and one animal in the FVIIai treatment group had a clot in the bronchus intermedius at post-mortem. Most animals in the two groups had some blood tinged secretions associated with suctioning at some point in the study. No severy or life-threatening bleeding complications occurred in either group.

Pulmonary and renal injury after TFPI infusion. To confirm the effects of TF blockade on ALI in E.coli sepsis, two baboons were treated with TFPI on the same experimental protocol. Activation of coagulation was blocked in sepsis after TFPI infusion with similar improvements in plasma fibrinogen levels. Terminal fibrinogen levels (t=48 h) in those animals were 75% and 95% of 12 h values. Like FVIIai, TFPI did not alter systemic hemodynamic parameters. Gas exchange and pulmonary mechanics were protected in both animals (see FIG. 2). Histopathology and fibrin immunostaining of lung tissue after TFPI showed decreased inflammatory cell infiltrates, decreased septal thickening, and decreased fibrin deposition in the lung. As in the FVIIai treated group, renal architecture was normal and fibrin staining in the kidneys was absent after TFPI.

TABLE 1

| Time (h) | 0 | 12 (13) | 18 | 24 | 36 | 48 | P value |
|---|---|---|---|---|---|---|---|
| Hgb | | | | | | | NS |
| Sepsis | 11.8 ± 0.4 | 11.2 ± 0.2 | 10.7 ± 0.5 | 10.7 ± 0.8 | 9.2 ± 0.5 | 7.8 ± 0.5 | |
| FVIIai | 11.7 ± 0.3 | 10.5 ± 0.5 | 10.0 ± 0.6 | 9.5 ± 0.6 | 9.7 ± 1.0 | 7.6 ± 0.7 | |
| Platelets | | | | | | | <0.001 |
| Sepsis | 180 ± 18 | 111 ± 18 | 46 ± 6 | 23 ± 3 | 17 ± 3 | 30 ± 7 | |
| FVIIai | 239 ± 16 | 148 ± 14 | 83 ± 14 | 38 ± 13 | 28 ± 8 | 22 ± 7 | |
| HR | | | | | | | NS |
| Sepsis | 101 ± 5 | 121 ± 8 | 139 ± 5 | 133 ± 6 | 134 ± 8 | 139 ± 9 | |
| FVIIai | 102 ± 4 | 122 ± 4 | 129 ± 5 | 131 ± 2 | 129 ± 5 | 127 ± 8 | |
| MAP | | | | | | | NS |
| Sepsis | 122 ± 6 | 114 ± 5 | 110 ± 4 | 112 ± 5 | 92 ± 6 | 88 ± 13 | |
| FVIIai | 118 ± 5 | 123 ± 7 | 98 ± 9 | 104 ± 7 | 98 ± 8 | 99 ± 10 | |
| CO/kg | | | | | | | NS |
| Sepsis | 0.16 ± 0.01 | 0.20 ± 0.02 | 0.24 ± 0.04 | 0.20 ± 0.02 | 0.20 ± 0.03 | 0.20 ± 0.02 | |
| FVIIai | 0.15 ± 0.01 | 0.24 ± 0.01 | 0.23 ± 0.02 | 0.24 ± 0.02 | 0.21 ± 0.01 | 0.25 ± 0.02 | |
| DO₂/kg | | | | | | | NS |
| Sepsis | 24.8 ± 1.8 | 28.4 ± 3.4 | 30.7 ± 4.0 | 24.8 ± 1.7 | 22.7 ± 2.5 | 19.6 ± 1.4 | |
| FVIIai | 22.1 ± 1.2 | 32.3 ± 2.2 | 28.2 ± 1.3 | 27.3 ± 1.1 | 25.8 ± 1.6 | 25.3 ± 3.9 | |
| VO₂/KG | | | | | | | NS |
| Sepsis | 5.5 ± 0.6 | 5.5 ± 0.6 | 6.2 ± 0.7 | 5.8 ± 0.3 | 5.4 ± 0.7 | 5.7 ± 0.4 | |
| FVIIai | 4.9 ± 0.5 | 6.6 ± 0.4 | 6.3 ± 0.3 | 5.6 ± 0.5 | 6.4 ± 0.7 | 4.6 ± 1.6 | |
| SVR/kg ?? | | | | | | | NS |
| Sepsis | 59642 ± 5070 | 45535 ± 4464 | 39310 ± 5412 | 44433 ± 6202 | 37993 ± 7913 | 32319 ± 6904 | |
| FVIIai | 62673 ± 5455 | 39367 ± 1939 | 33669 ± 4905 | 34734 ± 4473 | 35949 ± 5101 | 29137 ± 2233 | |
| PCWP | | | | | | | NS |
| Sepsis | 11 ± 1 | 12 ± 1 | 10 ± 1 | 11 ± 1 | 11 ± 1 | 11 ± 1 | |
| FVIIai | 10 ± 1 | 12 ± 1 | 10 ± 1 | 11 ± 1 | 12 ± 1 | 10 ± 1 | |
| V_{E} ?? | | | | | | | NS |
| Sepsis | 3.5 ± 0.2 | 3.4 ± 0.2 | 3.5 ± 0.3 | 4.0 ± 0.4 | 4.2 ± 0.6 | 4.8 ± 0.7 | |
| FVIIai | 3.5 ± 0.1 | 3.5 ± 0.1 | 4.1 ± 0.3 | 4.2 ± 0.3 | 4.4 ± 0.3 | 4.6 ± 0.3 | |
| FVIIai level | | | | | | | |
| FVIIai | 0 | 0 (8172 ± 879) | 4123 ± 650 | 3496 ± 385 | 2998 ± 164 | 2828 ± 118 | |

Table 1: Systemic measurements in sepsis control and FVIIai treated sepsis groups. Heat-killed bacteria were infused at t=0 hours and live bacteria were infused at t=12 hours. Data are shown as mean±sem and were analyzed with two-factor ANOVA. FVIIai drug levels in the treated group are shown in ng/ml plasma. Abbreviations: Temp (temperature, ° C.), Hgb (haemoglobin), V_{E} (minute ventilation, L/min), HR (heart rate), MAP (mean arterial pressure, mm Hg), CO (cardiac output, L/min), DO₂ (oxygen delievery, mL/min), VO₂ (oxygen comsumption, mL/min), SVR (systemic vascular resistance, dynes×cm×kg/10), PCWP (pulmonary capillary wedge pressure, mm Hg).

What is claimed is:

1. Use of modified FVII for the manufacture of a medicament for treatment of Acute Lung Injury (ALI) or Acute Respiratory Disease Syndrome (ARDS) in humans.

2. Use according to claim 1, for treatment of organ failure.

3. Use according to claim 2, wherein the organ is kidney, lung, adrenals, liver, small bowel, cardiovascular system, or haemostatic system.

4. Use according to claim 3, wherein the organ failure is failure of lung.

5. Use according to any one of claims 1 to 4, for maintaining or improving organ function.

6. Use according to claim 1, for treatment of pulmonary hypertension.

7. Use according to claim 1, for decreasing or minimizing procoagulant activity.

8. Use according to claim 7, wherein the procoagulant activity is associated with tissue factor expression by lung epithelial cells and tissue macrophages.

9. Use according to claim 1, for decreasing or minimizing inflammation.

10. Use according to claim 9, for decreasing or minimizing production of IL-6 and IL-8.

11. Use according to claim 1, for improving pulmonary gas exchange.

12. Use according to claim 1, for decreasing or minimizing lung oedema.

13. Use according to claim 1, for decreasing or minimizing lung protein leakage.

14. Use according to any of claims 1 to 13, wherein the modified FVII is FVII having at least one amino acid residue substitution, insertion, or deletion in the catalytic triad.

15. Use according to claim 14, wherein the modified FVII is FVII having at least one amino acid residue substitution, insertion, or deletion in positions Ser₃₄₄, Asp₂₄₂, and His₁₉₃.

16. Use according to claim 15, wherein the active site residue Ser₃₄₄ is modified, replaced with Gly, Met, Thr, or more preferably, Ala.

17. Use according to any of claims 1 to 13, wherein the modified FVII is FVIIa modified by reaction with a serine protease inhibitor.

18. Use according to claim 17, wherein the protease inhibitor is an organophosphor compound, a sulfanyl fluoride, a peptide halomethyl ketone, or an azapeptide.

19. Use according to claim 18, wherein the protease inhibitor is a peptide halomethyl ketone selected from Dansyl-L-Phe-Pro-Arg chloromethyl ketone, Dansyl-L-Glu-Gly-Arg chloromethyl ketone, Dansyl-L-Phe-Phe-Arg chloromethyl ketone and L-Phe-Phe-Arg chloromethylketone, Dansyl-D-Phe-Pro-Arg chloromethyl ketone, Dansyl-D-Glu-Gly-Arg chloromethyl ketone, Dansyl-D-Phe-Phe-Arg chloromethyl ketone and D-Phe-Phe-Arg chloromethylketone.

20. Use according to claim 19, wherein the protease inhibitor is D-Phe-Phe-Arg chloromethylketone.

21. Use of modified FVII for the manufacture of a medicament for preventing or minimizing chronic organ failure associated with ALI or ARDS in humans.

22. Use according to claim 21, wherein the ALI or ARDS is established before modified FVII is administered.

23. Use according to claim 21 or claim 22, wherein the organ failure is failure of kidney, lung, adrenals, liver, small bowel, cardiovascular system, or haemostatic system.

24. Use according to claim 23, wherein the organ failure is failure of lung.

25. Use according to any of claims 21 to 24, wherein the modified FVII is FVII having at least one amino acid residue substitution, insertion, or deletion in the catalytic triad.

26. Use according to claim 25, wherein the modified FVII is FVII having at least one amino acid residue substitution, insertion, or deletion in positions Ser₃₄₄, Asp₂₄₂, and His₁₉₃.

27. Use according to claim 26, wherein the active site residue Ser₃₄₄ is modified, replaced with Gly, Met, Thr, or more preferably, Ala.

28. Use according to any of claims 21 to 24, wherein the modified FVII is FVIIa modified by reaction with a serine protease inhibitor.

29. Use according to claim 28, wherein the protease inhibitor is an organophosphor compound, a sulfanyl fluoride, a peptide halomethyl ketone, or an azapeptide.

30. Use according to claim 29, wherein the protease inhibitor is a peptide halomethyl ketone selected from Dansyl-L-Phe-Pro-Arg chloromethyl ketone, Dansyl-L-Glu-Gly-Arg chloromethyl ketone, Dansyl-L-Phe-Phe-Arg chloromethyl ketone and L-Phe-Phe-Arg chloromethylketone, Dansyl-D-Phe-Pro-Arg chloromethyl ketone, Dansyl-D-Glu-Gly-Arg chloromethyl ketone, Dansyl-D-Phe-Phe-Arg chloromethyl ketone and D-Phe-Phe-Arg chloromethylketone.

31. Use according to claim 30, wherein the protease inhibitor is D-Phe-Phe-Arg chloromethylketone.

32. A method for treating Acute Lung Injury (ALI) or Acute Respiratory Disease Syndrome (ARDS) in humans, the method comprising administering to a patient in need of such treatment a therapeutically effective amount of modified FVII.

33. A method according to claim 32, wherein said patient is suffering from organ failure.

34. A method according to claim 33, wherein the organ failure is failure of one or more of kidney, lung, adrenals, liver, small bowel, cardiovascular system, or haemostatic system.

35. A method according to claim 34, wherein the organ failure is lung failure.

36. A method according to claim 32, wherein said treatment maintains or improves organ function.

37. A method according to claim 32, wherein said treatment improves pulmonary hypertension.

38. A method according to claim 32, wherein said treatment decreases procoagulant activity.

39. A method according to claim 38, wherein the procoagulant activity is associated with tissue factor expression by lung epithelial cells and tissue macrophages.

40. A method according to claim 32, wherein said treatment decreases inflammation.

41. A method according to claim 40, wherein said treatment decreases production of IL-6 and IL-8.

42. A method according to claim 32, wherein said treatment improves pulmonary gas exchange.

43. A method according to claim 32, wherein said treatment decreases pulmonary edema.

44. A method according to claim 32, wherein said treatment decreases lung protein leakage.

45. A method according to claim 32, wherein the modified FVII is FVII having at least one amino acid residue substitution, insertion, or deletion in the catalytic triad.

46. A method according to claim 45, wherein the modified FVII is FVII having at least one amino acid residue substitution, insertion, or deletion in positions Ser₃₄₄, Asp₂₄₂, and His₁₉₃.

47. A method according to claim 46, wherein the active site residue Ser₃₄₄ is modified, replaced with Gly, Met, Thr, or Ala.

48. A method according to claim 32, wherein the modified FVII is FVIIa modified by reaction with a serine protease inhibitor.

49. A method according to claim 48, wherein the protease inhibitor is an organophosphor compound, a sulfanyl fluoride, a peptide halomethyl ketone, or an azapeptide.

50. A method according to claim 49, wherein the protease inhibitor is a peptide halomethyl ketone selected from the group consisting of Dansyl-L-Phe-Pro-Arg chloromethyl ketone, Dansyl-L-Glu-Gly-Arg chloromethyl ketone, Dansyl-L-Phe-Phe-Arg chloromethyl ketone and L-Phe-Phe-Arg chloromethylketone, Dansyl-D-Phe-Pro-Arg chloromethyl ketone, Dansyl-D-Glu-Gly-Arg chloromethyl ketone, Dansyl-D-Phe-Phe-Arg chloromethyl ketone and D-Phe-Phe-Arg chloromethylketone.

51. A method according to claim 50, wherein the protease inhibitor is D-Phe-Phe-Arg chloromethylketone.

52. A method for preventing or minimizing chronic organ failure associated with ALI or ARDS in humans, the method comprising administering to a patient in need of such treatment an amount of modified FVII effective for preventing or minimizing chronic organ failure.

53. A method according to claim 52, wherein the ALI or ARDS is established before modified FVII is administered.

54. A method according to claim 52, wherein the organ failure is failure of kidney, lung, adrenals, liver, small bowel, cardiovascular system, or haemostatic system.

55. A method according to claim 54, wherein the organ failure is pulmonary failure.
